(19) United States
(12) Patent Application Publication
(10) Pub. No.: US 2019/0015073 A1
(43) Pub. Date: Jan. 17, 2019

(54) IMAGE DISPLAYING DEVICE, CONTROL METHOD THEREOF, AND MEDICAL DEVICE

(71) Applicant: SAMSUNG ELECTRONICS CO., LT, Suwon-si, Gyeonggi-do (KR)

(72) Inventors: Sung Hun PARK, Seoul (KR); Devadas PUTHUSSERIL, Kannur (IN)

(73) Assignee: SAMSUNG ELECTRONICS CO., LTD., Suwon-si, Gyeonggi-do (KR)

(21) Appl. No.: 16/067,273

(22) PCT Filed: Dec. 6, 2016
(86) PCT No.: PCT/KR2016/014243

§ 371 Date: Jun. 29, 2018

(30) Foreign Application Priority Data
Dec. 29, 2015 (KR) 10-2015-0188307

(51) Int. Cl. A61B 8/00; G06F 3/0482; G06F 3/0488; G06F 3/0481

(52) U.S. Cl. CPC A61B 8/465; G06F 3/0482; G16H 30/40; G06F 3/04886; G06F 3/04812; G06F 3/04883

(57) ABSTRACT

Provided is an image displaying device control method thereof, and medical device. A control method of an image displaying device may comprise displaying an image selector to select an image, displaying a plurality of section indicators around the image selector, selecting at least one section indicator from among the plurality of section indicators, and displaying an image corresponding to the image selector in a display section corresponding to the at least one section indicator.

### TECHNICAL FIELD

The present disclosure relates to an image displaying device, control method thereof, and medical device.

### BACKGROUND ART

An image displaying device refers to a device for displaying and providing various data, such as symbols, letters, numbers, figures, and pictures on the screen for the user. Such an image displaying device is also used in a medical field.

The image displaying device used in a medical field may display various data obtained by a specific method for doctors, nurses, medical technologists, examinees, caregivers, or other various users of the image displaying device. For example, the image displaying device assists a doctor, nurse, or the like, to make more accurate examination or diagnosis for an examinee by displaying an examination result or internal or external image of the examinee obtained by an in-vitro diagnostic medical device.

The image displaying device used in the medical field may be a desktop computer device or a medical device equipped with a displaying means such as a monitor device to display a captured image, e.g., a medical imaging diagnostic device. The medical imaging diagnostic device refers to a medical device capable of obtaining an image of an external portion or an internal portion of the examinee by using radiation, ultrasounds, or magnetic resonance to take the image.

### DISCLOSURE

### Technical Problem

An object of the present disclosure is to provide an image displaying device, control method thereof, and medical device capable of supporting an image desired by the user, e.g., a medical image, to be easily and quickly displayed.

Another object of the present disclosure is to provide an image displaying device, control method thereof, and medical device capable of reducing inconvenience in inputting user commands, which may be caused by increasing the size of a display means or a layout of the screen.

### Technical Solution

To attain the objects, the present disclosure provides an image displaying device, control method thereof, and medical device.

In accordance with an embodiment of the present disclosure, a control method of an image displaying device may comprise displaying an image selector to select an image, displaying a plurality of section indicators around the image selector, selecting at least one section indicator from among the plurality of section indicators, and displaying an image corresponding to the image selector in a display section corresponding to the at least one section indicator.

The displaying of the plurality of section indicators around the image selector may comprise displaying at least one of the plurality of section indicators around the image selector according to a position at which at least one display section corresponding to the at least one section indicator is placed.

The displaying of the plurality of section indicators around the image selector may comprise displaying the plurality of section indicators around the image selector when the image selector is selected.

The selecting of the at least one section indicator from among the plurality of section indicators may comprise displaying the at least one section indicator selected from among the plurality of section indicators differently from at least one other section indicator.

The displaying of the image selector to select an image may comprise displaying a plurality of image selectors to select different images.

The image selector may comprise at least one of letters, symbols, figures to stand for the selected image, and auxiliary images corresponding to the selected image.

The section indicator may be shaped to indicate a direction in which a corresponding one of the plurality of display sections is located.

The selecting of the at least one section indicator from among the plurality of section indicators may comprise at least one of selecting at least one of the plurality of section indicators by placing a cursor on the at least one section indicator, selecting at least one of the plurality of section indicators by touching or clicking the at least one section indicator, selecting at least one of the plurality of section indicators by manipulating an input means corresponding to each section indicator, and selecting at least one of the plurality of section indicators by making a gesture in a direction from the image selector to the at least one section indicator.

The selecting of the at least one section indicator from among the plurality of section indicators may comprise arbitrarily selecting at least one of the plurality of section indicators and entering a command to select the at least one section indicator arbitrarily selected.

The plurality of display sections may comprise sections capable of displaying at least one scout view.

In accordance with an embodiment of the present disclosure, an image displaying device may comprise a display configured to display an image selector to select an image and a plurality of section indicators around the image selector and a controller configured to control the display to display an image selected by the image selector in a display section corresponding to at least one of the plurality of section indicators when the at least one section indicator is selected.

The display may be configured to display at least one of the plurality of section indicators around the image selector according to positions at which at least one display section corresponding to the at least one section indicator is placed.

The display may be configured to display the plurality of section indicators around an image selector when the image selector is selected.

The display may be configured to display at least one section indicator selected from among the plurality of section indicators differently from at least one of non-selected section indicator.

The display may be configured to display a plurality of image selectors to select different images.

The image selector may comprise at least one of letters, symbols, figures to stand for the selected image, and auxiliary images corresponding to the selected image.

The section indicator may be shaped to indicate a direction in which a corresponding one of the plurality of display sections is located.

The at least one section indicator from among the plurality of section indicators may be selected by placing a cursor on the at least one section indicator or by touching or clicking the at least one section indicator or by manipulating an input means corresponding to each section indicator or by making a gesture in a direction from the image selector to the at least one section indicator.

The controller may be configured to move focus onto at least one of the plurality of section indicators based on user's manipulation.

The plurality of display sections may comprise sections capable of displaying at least one scout view.

### Advantageous Effects

According to the aforementioned image displaying device, control method thereof, and medical device, an image desired by the user may be displayed on a screen of the image displaying device only by simple manipulation without need for complicated manipulation or much action, thereby improving convenience in manipulating of the image displaying device.

According to the aforementioned image displaying device, control method thereof, and medical device, the user is able to manipulate an image to be displayed easily and quickly at an wanted point on the screen.

According to the aforementioned image displaying device, control method thereof, and medical device, the user is able to intuitively make one or two or more images displayed on the screen.

According, to the aforementioned image displaying device, control method thereof, and medical device, user inconvenience in inputting a command, which may be caused by increasing the size of the display means or by the screen layout, may be reduced.

### DESCRIPTION OF DRAWINGS

FIG. 1 shows an embodiment of a medical device;

FIG. 2 is a block diagram of an embodiment of a medical device;

FIG. 3 shows an embodiment of a screen displayed by a display;

FIG. 4 shows an example of a screen with a section indicator displayed thereon;

FIG. 5 shows an image selector and a section indicator in more detail;

FIG. 6 shows an example of a screen displayed when one section indicator is selected;

FIG. 7 shows an example of a screen displayed when one section indicator is selected by manipulation of an input means corresponding to each section indicator;

FIG. 8A shows an example of a screen with more cursors displayed thereon;

FIG. 8B shows an example of a screen displayed when a cursor is moved to the top of a section indicator;

FIG. 8C shows an example of a movement range of a cursor;

FIG. 9A shows an example of a screen when a section indicator touching gesture is input from an image selector;

FIG. 9B shows an example of providing, information regarding a display section corresponding to a selected section indicator for the user, when one section indicator is temporarily selected;

FIG. 10 shows a first example of a screen having an image displayed in a display section in response to selection of a section indicator;

FIG. 11 shows a second example of a screen having an image displayed in a display section in response to selection of a section indicator;

FIG. 12 shows an example of a screen with an image selector being a preview image;

FIG. 13 shows an example of a screen with a plurality of section indicators displayed around a preview image;

FIG. 14 shows an example of a screen displayed when one section indicator is arbitrarily or completely selected;

FIG. 15 shows another example of a screen displayed when one section, indicator is arbitrarily or completely selected;

FIG. 16 shows an example of a screen haying an image displayed in a display section in response to selection of a section indicator;

FIG. 17 is a first flowchart of a control method of an image displaying device, according to an embodiment of the present disclosure;

FIG. 18 is a second flowchart of a control method of an image displaying device, according to an embodiment of the present disclosure; and

FIG. 19 is a third flowchart of a control method of an image displaying device, according to an embodiment of the present disclosure.

### MODES OF THE INVENTION

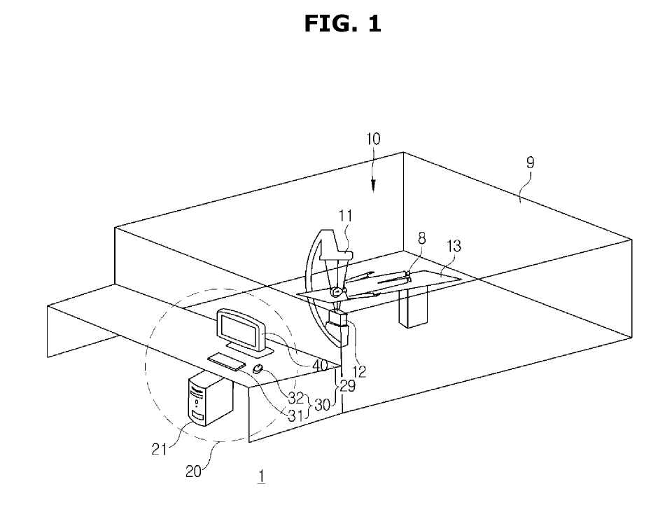

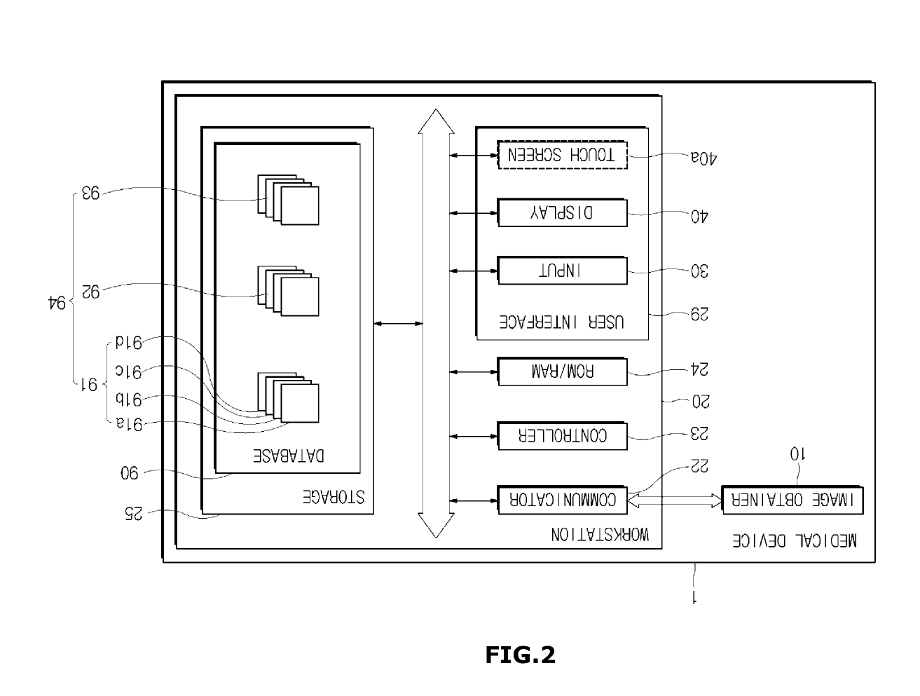

Referring to FIGS. 1 and 2, a medical device as an embodiment of an image displaying device will now be described.

FIG. 1 shows an embodiment of a medical device, and FIG. 2 is a block diagram of an embodiment of a medical device. FIGS. 1 and 2 show an embodiment of a medical imaging diagnostic device for the embodiment of the medical device.

As shown in FIGS. 1 and 2, a medical device 1 may include an image obtainer 10 for photographing an object 8 to obtain an image of an internal or external portion of the object 8, and a workstation 20 for performing various processes on the image obtained by the image obtainer 10 and visually providing the image for the user.

The image obtainer 10 and the workstation 20 may be implemented in a single mechanical device, or may each be implemented as a separate mechanical device, and in the latter case, the image obtainer 10 and the workstation 20 may be separated from each other. For example, the image obtainer 10 may be installed in an examination room 9 located separately, as shown in FIG. 1, and the workstation 20 may be installed outside the examination room 9.

In the case that the image obtainer 10 and the workstation 20 are installed to be separated from each other, the image obtainer 10 and the workstation 20 may be configured to send or receive mutual data or commands. Accordingly, the image obtained by the image obtainer 10 may be sent to the workstation 20, or various user commands input through the workstation 20 may be sent to the image obtainer 10.

Specifically, the image obtainer 10 and the workstation 20 may communicate with each other over a wired or wireless communication network. The wired communication network may be built with cables connected to the respective devices, and the cables may include pair cables, coaxial cables, fiber optic cables, or Ethernet cables. The wireless communication network may be implemented using a short-range communication network or a mobile communication network. The short-range communication network may be built using Wireless LAN (WLAN), Wi-Fi, Bluetooth, Zigbee, CAN communication, Wi-Fi Direct, Ultra Wideband, Infrared Data Association (IrDA), Bluetooth Low Energy (BLE), Near Field Communication (NFC), etc. The mobile communication network may be built based on various mobile communication standards, such as 3GPP, 3GPP2, air WiMax series.

The image obtainer 10 scans an internal or external portion of the object 8 based on radiation, ultrasound or magnetic resonance. The object 8 as herein used may include various subjects whose internal or external portions may be imaged by the image obtainer 10, such as the entire or a part of human body, an animal, or a plant.

For example, if the medical device is radiation imaging device, the image obtainer 10 may scan an internal portion of the object 8 using radiation. Specifically, the image obtainer 10 may include a radiation irradiator 11 for irradiating radioactive rays and a radiation detector 12 for receiving the radioactive rays that have penetrated the object, and the radiation irradiator 11 is configured to irradiate radioactive rays with certain energy, e.g., X-rays, to the object 8 according to an applied tube current and tube voltage. The radiation detector 12 is located at a position facing the radiation irradiator 11 with the object 8 as the center, and configured to receive radioactive rays that have penetrated the object 8 or radioactive rays around the object 8 and output an electric signal based on the received radioactive rays. In some embodiments, the image obtainer 10 may further include a holding table 13 to hold the object 8, in which case, the radiation detector 12 may be installed inside the holding table 13. Of course, the radiation detector 12 may also be installed outside the holding table 13.

In an embodiment, an electric signal output from the radiation detector 12 may be sent to a controller (not shown) included in the image obtainer 10, and the controller may create and correct an image based on the electric signal. In another embodiment, an electric signal output from the radiation detector 12 may be sent to a controller 23 included in the workstation 20, and the controller 23 may create and correct an image based on the electric signal output from the radiation detector 12.

In another example, if the medical device is an ultrasonic imaging device, the image obtainer 10 may irradiate ultrasounds to the inside of the object and receive ultrasounds reflecting off from the inside of the object or receive ultrasounds produced from the inside of the object The controller (not shown) included in the image obtainer or the controller 23 equipped in the workstation 20 may create an ultrasonic image by performing time-delaying and concentrating process on electric signals of a plurality of channels corresponding to the received ultrasounds.

In another example, if the medical device is a magnetic resonance imaging device, the image obtainer 10 may include a static magnetic field coil and an oblique magnetic field coil to produce magnetic fields around the object and an RF coil to receive pre-induction decay signal from the object, and the pre-induction decay signal received by the RF coil may be sent to the controller (not shown) included in the image obtainer 10 or the controller 23 equipped in the workstation 20, and the controllers may create a magnetic resonance image based on the pre-induction decay signal.

A radiological image, an ultrasonic image, or a magnetic resonance image created by the controller included in the image obtainer 10 or the controller 23 equipped in the workstation 20 may be sent to the storage 25, which may then store the radiological image, the ultrasonic image, or the magnetic resonance image.

The workstation 20 may perform various operations related to image acquisition and image processing and provide various data for the user or receive various user commands according to the user's manipulation.

The workstation 20 may be implemented by a general computer such as a desktop computer or a laptop computer, or may also be implemented by a computer device manufactured particularly for use of the medical device 1.

As shown in FIG. 2, the workstation 20 may include a communicator 22, a controller 23, a main memory device 24, a storage 25, and a user interface 29 in an embodiment. The communicator 22, the controller 23, the main memory device 24, the storage 25, and the user interface 29 may be provided to send or receive mutual data through circuits, conductor wires, or a wireless communication network provided in the device. Some of the communicator 22, the controller 23, the main memory device 24, the storage 25, and the user interface 29 may be provided in the image obtainer 10, or some of the communicator 22, the controller 23, the main memory device 24, the storage 25, and the user interface 29 may be provided in both the image obtainer 10 acid the workstation 20 if necessary.

The communicator 22 is provided to perform mutual communication with a communication module equipped in the image obtainer 10. Accordingly, the workstation 20 may receive various data related to the image obtained by the image obtainer 10 or may send various commands to the image obtainer 10. The communicator 22 may be implemented using a wired communication module or a wireless communication module depending on a method for communicating with the image obtainer 10. The wired communication module may be implemented using a communication port coupled with an extra cable, and a semiconductor chip and circuit for modulating, amplifying, or sending electric signals sent through the communication port. The wireless communication module may be implemented using an antenna to emit or receive radio waves, a communication chip to perform various processes on an electric signal received through the antenna, and other various pans required for wireless communication.

The controller 23 may control various operations related to the medical device 1. Specifically, the controller 23 may be configured to control general operation of the workstation 20, or control the image obtainer 10 by generating and outputting control, signals related to control over the image obtainer 10. In this case, the controller 23 may control operation of each part of the workstation 20 by generating and sending a control signal to the part of the workstation 20 through a circuit or cable. Furthermore, the controller 23 may control general operation of the image obtainer 10 by sending a certain control signal to the image obtainer 10 through the communicator 22.

Specifically, for example, the controller 23 may control an image to be created based on data received from the image obtainer 10 through the communicator 22, control the image to be corrected, or control the storage 25 to stare the created or corrected image. Furthermore, the controller 23 may make at least one of the image obtainer to and the workstation 20 operate according to the user's command input through the input 30. In addition, the controller 23 may send a control signal to the display 40 to control the display 40 to display an image created based on the data received from the image obtainer 10, a graphic user interface (GUI), or other various images.

The main memory device 24 may temporarily or non-temporarily store various data required for operation of the controller 23 to assist with operation of the controller 23. The main memory device 24 may include a storage medium, such as a ROM or a RAM. The RAM may include e.g., a dynamic RAM (DRAM) or a static RAM (SRAM). The ROM may include e.g., an erasable and programmable ROM (EPROM), an electrically erasable and programmable ROM (EEPROM) or a mask-ROM.

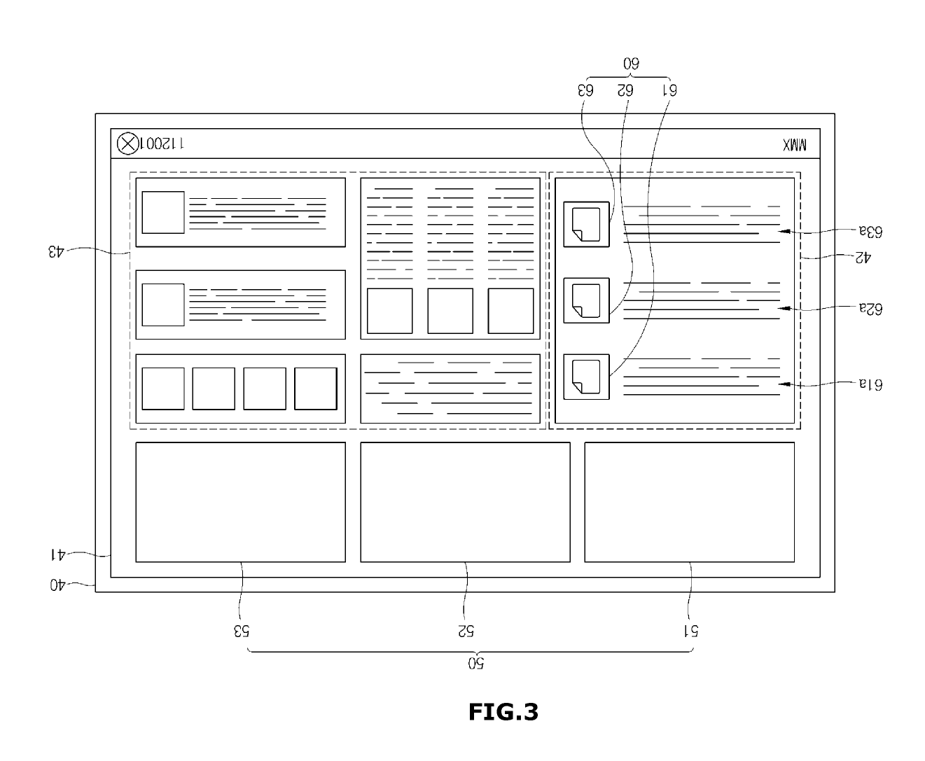

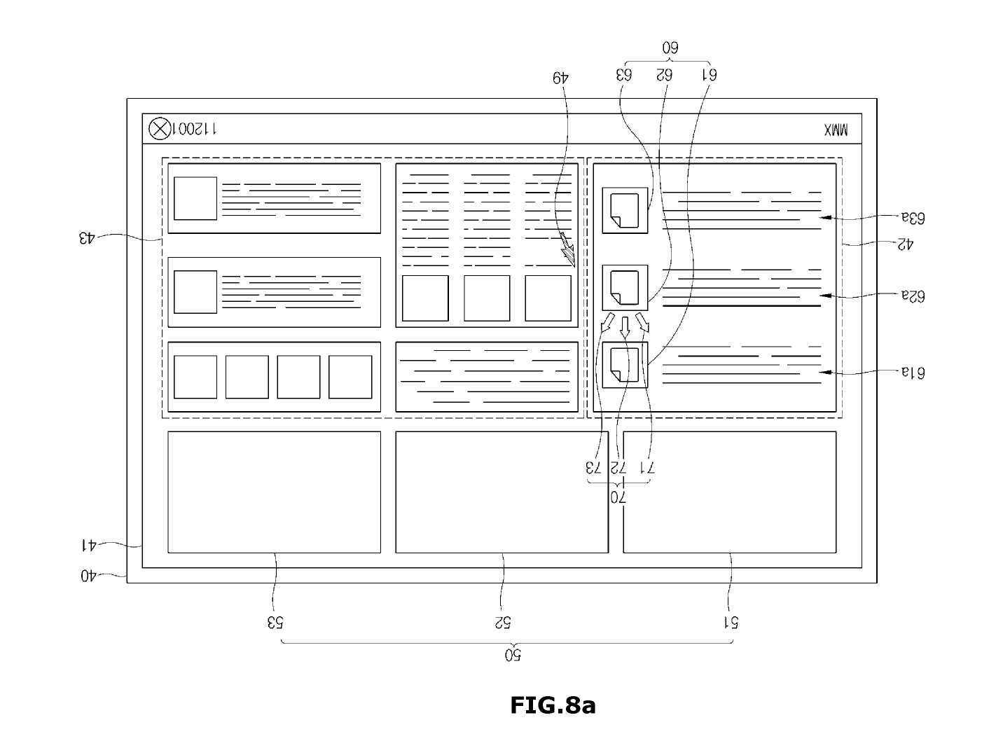

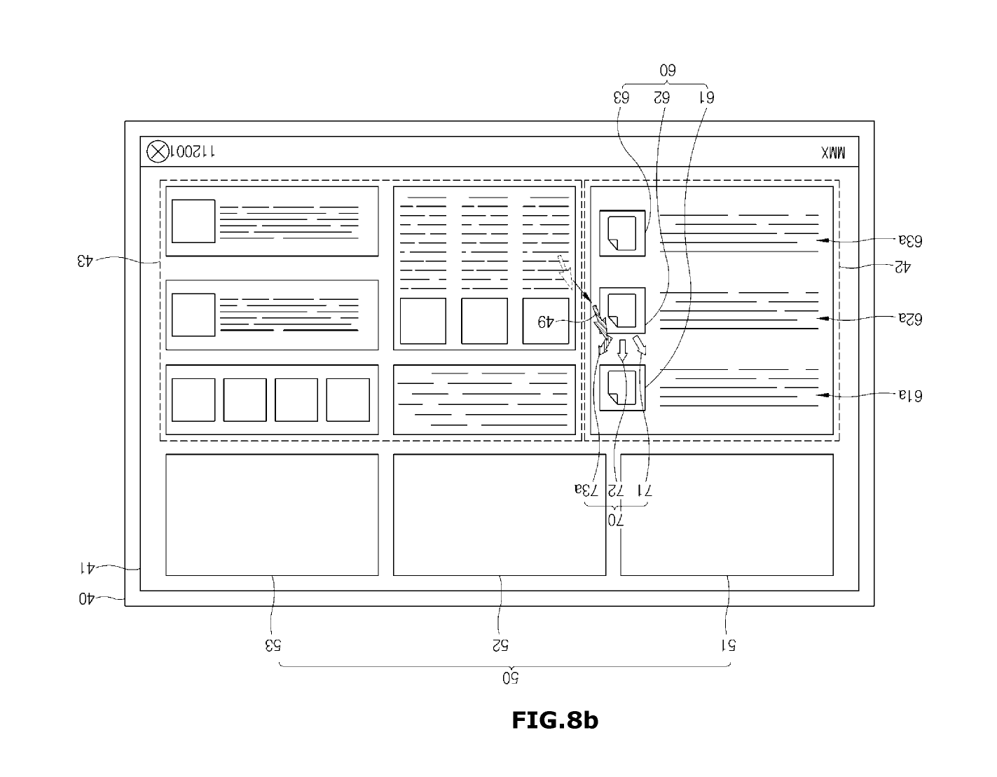

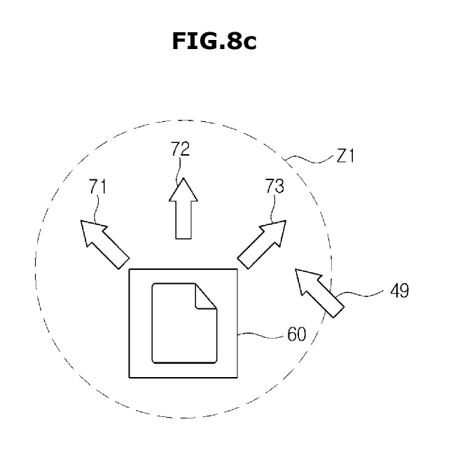

The storage 25 may temporarily or non-temporarily store various data required for operation of the controller 23, images obtained from the image obtainer 10, a GUI displayed on the display 40, or various image data to realize other various images. The various image data as herein used may include data about frame forms to be displayed on the display 40 or many different types of graphic objects to be displayed on the display 40, and the data about many different types of graphic objects may include data for features of icons or buttons, data for the feature of a cursor 49 (see FIGS. 8A and 8B), various character data, or the like. The cursor 49 represents an indicator such as a figure or a picture to indicate an input position on the screen 41 (see FIG. 3).

The storage 25 may be implemented using a solid state drive (SSD) for storing information using a semiconductor or a hard disc drive (HDD) for storing information using a magnetic disc. In addition, the storage 25 may be implemented using various storage media, such as compact disc (CD), laser disc (LD), magneto-optical disc or floppy disc, capable of storing information. Furthermore, the storage 25 may be implemented using various media capable of storing a database 90 and the like.

In the storage 25, as shown in FIG. 2, the database 90 comprised of one or multiple images 94 may be stored. The images 94 forming the database 90 may include images obtained by the image obtainer 10. The multiple images 94 stored in the database 90 may be variously classified and stored according to a predetermined classification method. The multiple images 94 may be classified by type of the obtained image, for example, may be classified by condition at the time of scanning, by processing method for various data obtained by scanning, or by shape or direction of an object appearing in the image.

For example, the medical device 1 may be a magnetic resonance imaging (MRI) device or a computed tomography (CT) device, and the image obtainer 10 may obtain data corresponding to the respective images 94 using an MRI scanning method or CT scanning method. In this case, images 94 stored in the database 90 may be classified into first images 91, second images 92, and third images 93.

In an embodiment, the first, second, and third images 91, 92, and 93 may be scout views. A scout view refers to an image used to determine a point to be scanned in advance to correctly scan a cross-section of a target portion while in CT scanning. In this case, the first images 91 may include sagittal plane images, the second images 92 may include coronal plane images, and third images 93 may include axial plane images. The sagittal plane refers to a section of a human body or of a part of the human body from the lateral direction, the coronal plane refers to a section of a human body or of a part of the human body from the frontal direction, and the axial plane refers to a section of a human body or of a part of the human body from the upper direction.

In an embodiment, a plurality of images 91a to 91d may be equally classified and stored in the database 90. For example, as shown in FIG. 2, the plurality of images 91a to 91d may be classified into the first images 91. In this case, the plurality of images 91a to 91d may be a set of images related to one another. For example, the plurality of images 91a to 91d may be the same sectional images of the object, e.g., sagittal plane images, obtained by sequential scanning.

In this case, each of the plurality of images 91a to 91d may be multiple image frames of a moving image, and the multiple image frames may be sequentially reproduced on the display 40 according to the user's manipulation or what is determined in advance. Accordingly, the user may watch the moving image of a particular section of the object, e.g., of the sagittal plane.

The aforementioned communicator 22, controller 23, main memory device 24, and storage 25 may be installed inside a main body 21 of the workstation 20, for example, installed directly on a certain substrate or formed into a certain module and installed on a substrate provided inside the main body 21.

The user interface 29 may include an input 30 and a display 40. At least one of the input 30 and the display 40 of the user interface 29 may be arranged separately from the main body 21 or installed directly in the main body 21 depending on embodiments. For example, the display 40 of the user interface 29 may be integrated with the main body 21 or may be physically separated from the main body 21 but may be able to communicate with the main body 21 via conductor wires.

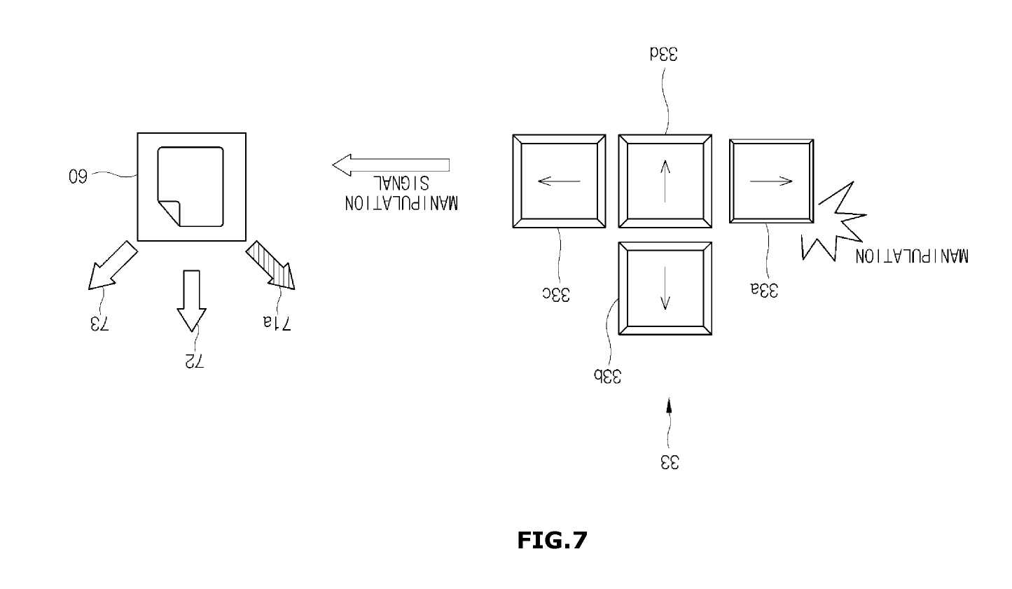

The input 30 may receive various commands or information from the user. Specifically, the input 30 may output an electric signal corresponding to the user's manipulation, and the electric signal is sent to the controller 23 through a circuit or cable. The input 30 may be implemented using at least one of a mechanical button, a touch pad, a knob, a joy stick, a trackball, a track pad and a mouse device 32, and the mechanical button may include at least one of a keyboard device 31 with predetermined buttons arranged thereon in a predetermined pattern, a keypad and a direction indicator button, e.g., direction keys 33 (see FIG. 7).

The display 40 may provide various visual information for the user. Specifically, the display 40 may display at least one of an image captured according to a control signal from the controller 23, a GUI, and other various images. The display 40 may be implemented using a cathode ray tube (CRT), or various types of display panel for displaying certain characters, symbols, or images, such as a liquid crystal display (LCD) panel, a light emitting diode (LED) display panel, an organic light emitting diode (OLED) display panel, an active-matrix organic light emitting diode (AMOLED) display panel, or a cold cathode fluorescent lamp.

In some embodiments, the user interface 29 may include a touch screen 40a. The touch screen 40a refers to a means for receiving user commands according to the user's touch gesture while displaying a certain image for the user. In an embodiment, the touch screen 40a is configured to detect various touch gestures made by the user. For example, the touch screen 40a is configured to detect various touch gestures, such as tapping, double tapping, dragging, swiping, etc. The touch screen 40a may be implemented with a resistive membrane type touch screen, a capacitive type touch screen, an infrared type touch screen, or an ultrasonic type touch screen depending on embodiments. With the touch screen 40a, at least one of the input 30 and the display 40 may or may not be omitted. If the input 30 is not omitted, the workstation 20 may use one or both of operation of the input 30 and touch operation on the touch screen 40a to receive a user command from the user.

Referring to FIGS. 3 to 15, various embodiments of a method for displaying at least one image in a display section 50 on a screen 41 by manipulating at least one of the screen 41 displayed on the display 40, the input 30, and the touch screen 40a will now be described.

FIG. 3 shows an embodiment of a screen displayed by a display.

Referring to FIG. 3, the display 40 may display images in certain symbols, text and/or figures on the screen 41.

In an embodiment, according to the user's manipulation, there are a display section 50 in which certain images are displayed, an image selecting section 42 in which various figures or text is displayed to select an image, and an auxiliary display section 43 to display other various information arranged on the screen 41.

In the display section 50, one or two or more images may be displayed, and the images to be displayed include images obtained by the image obtainer 10. A plurality of images may be displayed in the display section 50 at the same time, and for this, the display section 50 may include a plurality of display sections 51 to 53 to display certain images. In the following description, for convenience of explanation, the display, sections will be referred to as a first display section 51, a second display section 52, and a third display section 53 from the left of the screen 41. The first, second, and third display sections 51, 52, and 53 may display the same image or may display different images. In this case, it is possible that two of the first, second, and third display sections 51, 52, and 53 display the same image while the other one displays a different image from the image displayed in the two display sections and that each of the first, second, and third display sections 51, 52, and 53 displays a different image. The first, second, and third display sections 51, 52, and 53 may also display still images or moving images. In this case, one or two of the first, second and third display sections 51, 52, and 53 may display still images and the other display section(s) may display moving images.

The image selecting section 42 may have at least one image selector 60 displayed therein to select an image and further have at least one text, number, or symbol 61a, 62a, and 63a displayed therein. The at least one text, number, or symbol may make up a word or sentence corresponding to at least one image selector 60, and the word or sentence corresponding to the at least one image selector 60 may include a word or sentence to explain e.g., content of the at least one image selector 60. Details of the image selector 60 will be described later.

The auxiliary display section 43 may have information for controlling the medical device 1, controlling an information processing device 20, or identifying the object 8, or various information required by the user displayed thereon. For example, information about whether the medical device 1 is operating, information about various settings regarding scanning by the medical device 1, and/or various information such as status of the information processing device 20, identification number, birth year, month, and date, sex, medical record, scanning, time or scheduled scanning time of the object 8 may be displayed in the auxiliary display section 43. Furthermore, an image for controlling, the medical device 1 or the information processing device 20 may be displayed in the auxiliary display section 53 as required, and the user may input his/her command by touching or clicking the displayed image using the mouse device 32.

The image selector 60 will now be described in detail.

The image selector 60 is provided to select and call a certain image according to the user's selection. The user may select an image to be displayed in the display section 50 on the screen 41 by selecting the image selector 60.

The image selector 60 may be implemented using text, symbols or various figures, and for example, may be implemented in the form of an icon. The image selector 60 may have a shape from which the user may intuitively know that the image selector 60 is what provides an image selecting function. The image selector 60 may be designed to be shaped like e.g., paper with a folded corner, as shown in FIG. 3. This is, however, by way of example, and the image selector 60 may be designed in various forms that may be considered by the designer.

When the image selector 60 is selected according to the user's manipulation, an image corresponding to the selected image selector 60 is called out. In this case, the user may select the image selector 60 by, for example, directly touching once or multiple times on. the image selector 60, or by manipulating the keyboard device 31, the mouse device 32, the direction key 33, or the like to move a cursor 49 onto the image selector 60. Furthermore, the user may move the cursor 49 to overlap the image selector 60 by manipulating the keyboard device 31, the mouse device 32, or the direction key 33, and then select the image selector 60 overlapping with the cursor 49 by clicking a certain button, e.g., the left or right button of the mouse device 32 once or multiple times (clicking or double-clicking). Once the image selector 60 is selected, an image corresponding to the selected image selector 60 may be displayed in one of the plurality of display sections 51 to 53.

In an embodiment, once the image selector 60 is selected, an electric signal is sent to the controller 23 from the user interface 29, and the controller 23 may call an image, e.g., the first image 91, corresponding to the image selector 60, which is selected from the storage 25 according to the electric signal. In this case, the first image 91 called out or information to indicate the first image 91 may be stored in the main memory device 24. In some embodiments, the controller 23 may call a plurality of images, e.g., a plurality of first imams 91a to 91d, and store related information in the main memory device 24.

The image selector 60 may include a plurality of image selectors 61 to 63 as shown in FIG. 3, and the plurality of image selectors 61 to 63 may be displayed in the image selecting section 42 in a predetermined configuration. For example, the plurality of image selectors 61 to 63 may be sequentially arranged from top to bottom or from bottom to top in the image selecting section 42, or from left to right or from right to left in the image selecting section 42.

The user may select one image selector, e.g., the first image selector 61, from among the plurality of image selectors 61 to 63, and once the first image selector 61 is selected, an image corresponding to the first image selector 61, e.g., the first image 91, may be called out. In the same way as described above, the user may select one of the plurality of image selectors 61 to 63 by touching once or multiple times or by manipulating the keyboard device 31, mouse device 32, or direction key 33.

When one of the plurality of image selectors 61 to 63 is selected, the user interface 29 may output and send an electric signal to the controller 23, and the controller 23 may call an image from the storage 25 corresponding to the selected image selector.

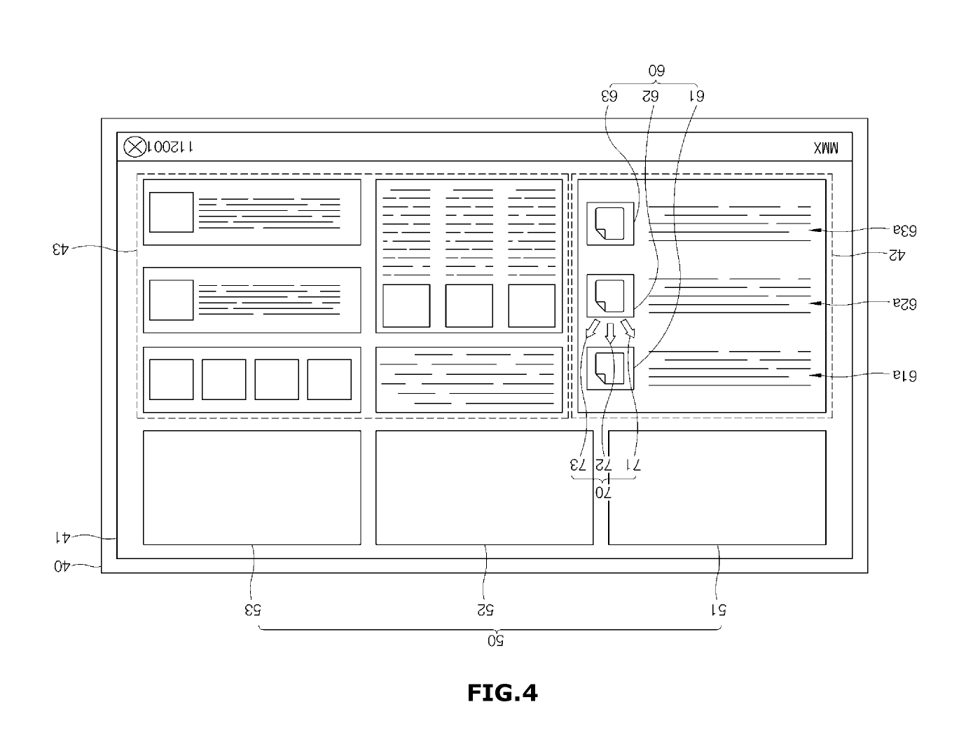

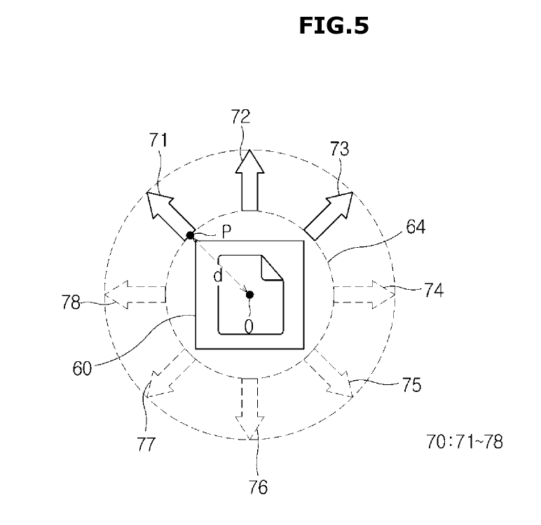

FIG. 4 shows an example of a screen with a section indicator displayed thereon, and FIG. 5 shows an image selector and a section indicator in more detail.

In an embodiment, once the image selector 60 to 63 is selected, at least one section indicator 70 may be displayed around the selected image selector 60 to 63. For example, if the first image selector 61 to call the first image 91 is selected.

at least one section indicator 70 may be displayed around the first image selector 61, if the second image selector 62 to call the second image 92 is selected as shown in FIG. 2, at least one section indicator 70 may be displayed around the second image selector 62, and if the third image selector 63 to call the third image 93 is selected, at least one section indicator 70 may be displayed around the third image selector 63.

The section indicator 70 is displayed on the screen 41 to select a particular display section 51 to 53 with an image displayed therein. Specifically, in response to selection of the section indicator 70, an image selected by the image selector 60 may be displayed in the display section 50 corresponding to the selected section indicator 70.

The section indicator 70 may be implemented as a graphic object in a form that allows the user to intuitively know the particular display section 51 to 53 with the image displayed therein. For example, the section indicator 70 may have the form, e.g., an arrow form, that indicates a direction in which the corresponding display section 51 to 53 is arranged, as shown in FIG. 4. Alternatively, the section indicator 70 may have the form of a certain figure, such as a triangle or a rectangle that indicates a direction in which the corresponding display section 51 to 53 is arranged. Furthermore, the section indicator 70 may be implemented as a thumbnail image to indicate the corresponding display section 51 to 53, and may have the figure of a letter or a number, e.g., 1, 2, 3, etc., to identify the corresponding display section 51 to 53. In the latter case, the number 1 may indicate the first display section 51, the number may indicate the second display section 52, and the number 3 may indicate the third display section 53.

As shown in FIG. 5, the section indicator 70 may be displayed in a surrounding area 64 of the image selector 60. In an embodiment, the section indicator 70 may be displayed to overlap with the image selector 60 or to be close to the image selector 60. For example, the entire or part of the section indicator 70 may overlap the image selector 60 so that the section indicator 70 and the image selector 60 may be displayed to be adjacent 3 each other. In another example, the section indicator 70 may be displayed a certain distance d away from the image selector 60. In the case that the image selector 60 and the section indicator 70 are displayed a distance away from each other, the image selector 60 and the section indicator 70 may be placed and displayed at first and second points pl and o, respectively, which are the certain distance d away from each other. In this case, the image selector 60 and the section indicator 70 are arranged at the points p and o, respectively, which may prevent selection of a particular section indicator, e.g., selection of the first section indicator 71, from being interfered by other section indicators 72 to 78 and prevent the user manipulation on the input 30 from exceeding a certain extent. In a specific example, the image selector 60 and the section indicator 70 may be arranged and displayed such that a distance moved by the cursor 49 according to manipulation of the mouse device 32 or a distance moved by a touch means across the touch screen 40a does not exceed a certain range. In this case, the distance d between the point p which one of the section indicators 70 is displayed and the point o at which the image selector 60 is displayed may be set such that the distance moved by the cursor 49 or the distance moved by the touch means may be minimized or be less than a certain value. In this way, with the image selector 60 and the section indicator 70 displayed to be adjacent to each other, the user may quickly and easily select the section indicator 70 with the least manipulation.

In some embodiments, the distance d between a position at which one of the section indicators 70 is displayed and the image selector 60 may or may not be equally applied for each section indicator 71 to 78. For example, a distance between a particular section indicator, e.g., the first section indicator 71 or the third section indicator 73 and the image selector 60 may be longer or shorter than a distance between the second section indicator 72 and the image selector 60.

In an embodiment, the number of the section indicators 70 displayed around the image selector 60 may correspond to the number of images to be displayed at the same time in the display sections 50. For example, the number of the section indicators 70 may be the same as the number of the display sections 50, and as shown in FIG. 3, when there are three display sections 51 to 53, there may be three section indicators 71 to 73 as well. If there is only one display section 50, only one section indicator 70 may be displayed around the image selector 60.

The section indicators 70 may be provided to correspond to the plurality of display sections 50 one on one. Different section indicators 70 correspond to different display sections 50, and different display sections 50 may correspond to different section indicators 70. For example, the first section indicator 71 among the plurality of section indicators 71 to 73 may be provided to correspond to the first display section 51 among the plurality of display sections 51 to 53, the second section indicator 72 may be provided to correspond to the second display section 52, and the third section indicator 73 may be provided to correspond to the third display section.

The plurality of section indicators 71 to 78 corresponding to the display sections 51 to 53 may be displayed at particular positions around the selected image selector 60 to correspond to positions at which the display sections 51 to 53 are arranged. For example, as shown in FIGS. 3 and 4, when the first display section 51 is provided in the upper left corner of the screen 41, the first section indicator 71 corresponding to the first display section 51 may be displayed in the upper left side of the image selector 60 according to the position of the first display section 51. When the second display section 52 is provided in the upper center of the screen 41. the second section indicator 72 corresponding to the second display section 52 may be displayed in the upper center of the image selector 60 according to the position of the second display section 52. When the third display section 53 is provided in the upper right corner of the screen 41, the third section indicator 73 corresponding to the third display section 53 may be displayed in the upper left side of the image selector 60 to match the position of the third display section 53.

Likewise, if a display section (not shown) is provided in the left side of the screen 41, the section indicator 74 corresponding to the display section provided in the left side of the screen 41 may be displayed in the right side of the image selector 60, and if a display section (not shown) is provided in the lower center of the screen 41, the section indicator 76 corresponding to the display section may be displayed in the lower side of the image selector 60. In addition, depending on the positions of the display sections corresponding to the section indicators 71 to 78, the section indicators 71 to 78 may be displayed at particular positions around the image selector 60.

In other words, if the image selector 60 is selected, as many section indicators 70 as the number of the display sections 50 may be displayed around the image selector 60 at the corresponding position of the display section 50.

Furthermore, in an embodiment, the number of the section indicators 70 may be less than the number of display sections 50. In this case, there are no corresponding section indicators set for some of the display sections 50, and a display section for which there is a corresponding section indicator may display an image without manipulation of the section indicator 70 but according to a predetermined setting or the user's other manipulation. For example, in a display section for which there is no corresponding section indicator, a certain image selected by e.g., the controller 23, may be displayed regardless of the user's manipulation. In another example, in a display section for which there is no corresponding section indicator, a certain image may be displayed without selection of the section indicator 70 but according to manipulation of a button.

When at least one section indicator 70 is displayed around the selected image selector 60, the user may select at least one of the section indicators 70 or perform directional manipulation corresponding to the direction in which the at least one section indicators 70 is arranged around the image selector 60, thereby enabling the image 94 corresponding to the selected image selector 60 to be displayed on at least one display screens 51 to 53.

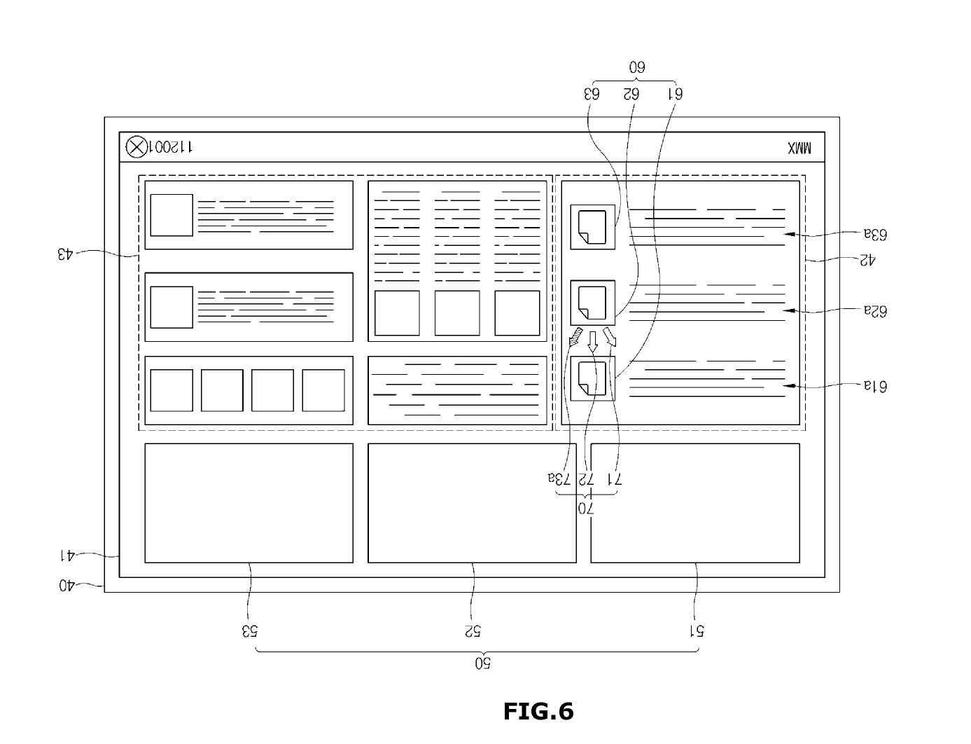

FIG. 6 shows an example of a screen displayed when one section indicator is selected.

As shown in FIG. 6, a section indicator, e.g., a third section indicator 73a, may be changed in shape or color according to the user's manipulation, and accordingly, e.g., the third section indicator 73a may be displayed on the screen 41 differently from other section indicators, e.g., the first and second section indicators 71 and 72 according to the user's manipulation.

Specifically, a section indicator, the third section indicator 73a may be displayed in different color or shape from other section indicators, e.g., the first and second section indicators 71 and 72. For example, if the first to third section indicators 71 to 73 are displayed in white arrows, the third section indicator, according to the user's manipulation, may be displayed in a red arrow. If the first to third section indicators 71 to 73 are displayed in a certain size of arrows, the third section indicator, according to the user's manipulation, may be displayed in a relatively larger size of arrow.

In this case, if the user arbitrarily or completely selects a section indicator, the section indicator may be displayed differently from the other section indicators, e.g., the first and second section indicators 71 and 72. The arbitrary selection means that the user's selection is not yet confirmed as in the case that the cursor 49 is displayed to overlap the section indicator or that a focus is placed on the section indicator, and the complete selection means that the user's selection of the section indicator is completed by the user's double-clicking with the mouse or by inputting a confirmation command while the focus is placed. The focus as herein used refers to a means for indicating at least one of a plurality of items if there exist the plurality of items. The user may place the focus on one of the plurality of items and select the item indicated by the focus by inputting an extra command to select the item indicated by the focus. In some embodiments, at least one of the arbitrary selection and the complete selection may be performed by directional manipulation. The directional manipulation as herein used means manipulation to indicate a particular direction, and may include various manipulation behaviors that may be considered by the designer, such as e.g., manipulation of a particular direction key, manipulation of a mouse to move the cursor in a particular direction, or a touch gesture like touching a point and then dragging to another point.

In an embodiment, when a section indicator, e.g., the third section indicator 73 is arbitrarily selected by the user, the third section indicator 73a may be changed and displayed differently from the other section indicators 71, 72 to let the user know that the third section indicator 73a is arbitrarily selected. Afterwards, the user may complete selection of the indicator 73a by additional manipulation of selecting the third section indicator 73a, e.g., by performing button-click manipulation on the mouse device 32 or additional touching of the third section indicator 73a to completely select the third section indicator 73. When the selection is completed, the third section indicator 73a selected by the user may continue to be differently displayed from the other section indicators 71, 72.

In another embodiment, when a section indicator, e.g., the third section indicator 73, is completely selected by the user and thus the selection of the section indicator, e.g., the third section indicator 73, is completed, the third section indicator 73a is changed and displayed differently from the other section indicators 71, 72. Accordingly, the user may be informed that the third section indicator 73a is completely selected. Before a section indicator, e.g., the third section indicator 73 is completely selected, the third section indicator 73 may have been displayed in the same way as the other section indicators 71, 72 or may have been displayed differently from the other section indicators 71, 72 due to the arbitrary selection as described above. While the third section indicator 73 is arbitrarily selected and accordingly, displayed differently from the other section indicators 71, 72, when the third section indicator 73 is completely selected, the third section indicator 73 may be displayed identically to or differently from an occasion when the third section indicator 73 is arbitrarily selected. In other words, the third section indicator 73 selected completely may be displayed differently from not only the other section indicators 71 72 but also the arbitrarily selected third selection indicator 73.

Various embodiments of selecting one of the plurality of section indicators 71 to 73 will now be described.

FIG. 7 shows an example of a screen displayed when one section indicator is selected by manipulation of an input means corresponding to each section indicator.

In an embodiment, when the plurality of section indicators 71 to 73 are displayed around the image selector 60, the user may arbitrarily or completely select one of the plurality of section indicators 71 to 73 by manipulating a certain mechanical button, e.g., the direction key 33, as shown in FIG. 7. The direction key 33 may include a plurality of keys 33a to 33d indicating different directions, and the keys 33a to 33d may correspond to the section indicators 71 to 73, and according to manipulation of one of the keys 33a to 33d, a corresponding section indicator 71 to 73 may be selected. In this case, for example, of the plurality of keys 33a to 33d, a left direction key 33a may correspond to the first section indicator 71 displayed in the leftmost side, an upper direction key 33b may correspond to the second section indicator 72 displayed in the center, and a right direction key 33c may correspond to the third section indicator 73 displayed in the rightmost side. Furthermore, the lower direction key 33d may be for inputting a command to cancel the arbitrary selection. Configurations of the section indicators 71 to 73 corresponding to the respective keys 33a to 33d are only by way of example, and may be variously defined by the designer. The user may manipulate one of the plurality of keys 33a to 33d, e.g., the left direction key 33a, to arbitrarily or completely select one of the plurality of section indicators 71 to 73, e.g., the first section indicator 71 or cancel the selection. As described above, the section indicator arbitrarily or completely selected, e.g., the first section indicator 71, may be displayed differently from the other section indicators 72, 73.

FIGS. 8A and 813 show an example of a screen displayed when a cursor is moved to the op of a section indicator. In FIGS. 8A and 8B, art example of selecting the second image selector 62 from among the plurality of image selectors 61 to 63 using the cursor and selecting the third section indicator 73 from among the plurality of section indicators 71 to 73 is shown.

In an embodiment, as shown in FIGS. 8A and 8B, when the plurality of section indicators 71 to 73 are displayed, around the image selector 60, e.g., the second image selector 62, the user may manipulate the mouse device 32 to move the cursor 49 across the screen 41 and arbitrarily or completely select one of the section indicators 71 to 73, e.g., the third section indicator 73.

In this case, the user may arbitrarily or completely select the third section indicator 73 by manipulating the mouse device 32 to move the cursor 49 onto one of the section indicators 71 to 73, e.g., the third section indicator 73. Furthermore, the user may arbitrarily or completely select one of the plurality of section indicators 71 to 73, e.g., the third section indicator 73 by moving the cursor 49 to the third section indicator 73 and clicking, a button, e.g., the left button or right button of the mouse device once or multiple times.

If one of the plurality of section indicators 71 to 73, e g the third section indicator 73 is arbitrarily selected, the user may completely select the third section indicator 73 through additional manipulation, e.g., by, clicking a button, e.g., the left button or right button of the mouse device 32 once or multiple times.

Furthermore, the user may select one of the section indicators 71 to 73 by dragging operation of the mouse device 32. Specifically, the user may place the cursor 49 on a selected image selector, e.g., the second image selector 62, click the left or right button of the mouse device 32, and then move the mouse device 32 from the second image selector 62 to one of the plurality of section indicators 71 to 73, e.g., the third section indicator 73 to arbitrarily or completely select the third section indicator 73.

As described above, the section indicator arbitrarily or completely selected by manipulation of the mouse device 32 and movement of the cursor 49, e.g., the third section indicator 73, may be displayed differently from the other section indicators, the first and second section indicators 71, 72.

FIG. 8C shows an example of a movement range of a cursor.

As shown in FIG. 8C, in the case that the user manipulates the mouse device 32 to move the cursor 49, it may be configured for one of the plurality of section indicators 71 to 73 to be selected provided the cursor 49 is moved within a certain range z1. For example, if the user manipulates the mouse device 32 to place the cursor 49 within the certain range z1, it may be configured for one of the plurality of section indicators 71 to 73 to be selected based on the position of the cursor 49, and if the user manipulates the mouse device 32 to place the cursor 49 out of the certain range z1, it may be configured for no one of the plurality of section indicators 71 to 73 to be selected. In other words, in the case that one of the section indicators 71 to 73 is selected, if the cursor 49 is moved out of the predetermined range z1, the selection of the section indicator 71 to 73 may be canceled or otherwise if the cursor 49 enters the predetermined range z1, one of the plurality of section indicators 71 to 73 may be selected.

Although an example of selecting the second image selector 62 and then the third section indicator 73 by manipulation of the mouse device 32 has been described, this may be equally or with arbitrary modification by the designer, applied to an occasion when other image selector 61, 62 and other section indicator 71, 72 are selected.

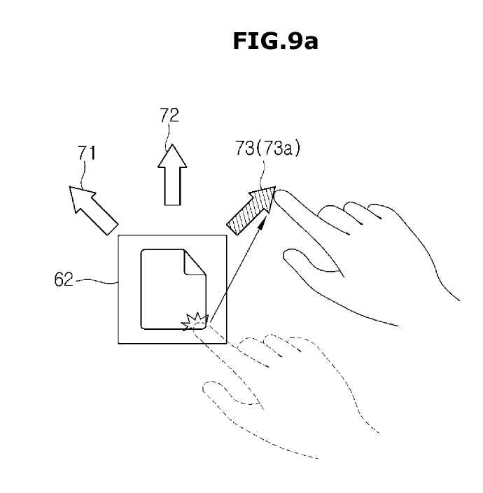

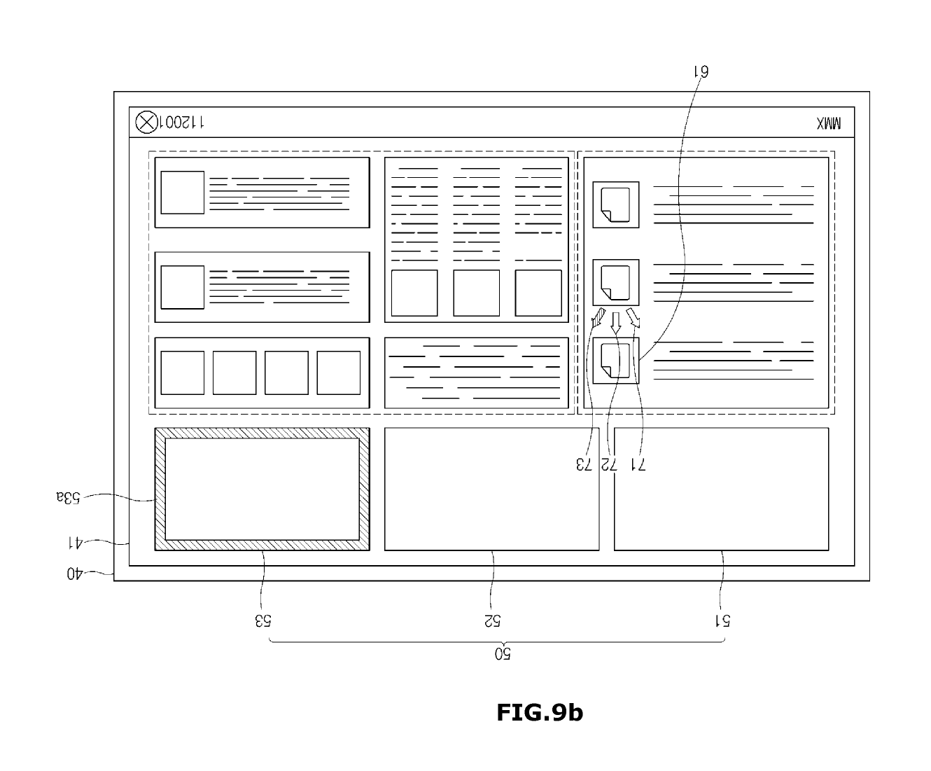

FIG. 9A shows an example of a screen when a touch gesture is made in the direction to a section indicator from an image selector. In FIG. 9, an example of selecting the second image selector 62 from among the plurality of image selectors 61 to 63 displayed on the touch screen 40a and selecting the third section indicator 73 from among the plurality of section indicators 71 to 73 is shown.

In an embodiment, in a case that the touch screen 40a is equipped in the workstation 20, the user may select one of the plurality of section indicators 70 displayed around the selected image selector 60 by making a certain touch gesture on the touch screen 40a.

For example, in a case that the touch screen 40a displays the plurality of section indicators 71 to 73 around an image selector, e.g., the second image selector 62, as shown in FIG. 9, the user may make a touch gesture on the touch screen 40a to enter a command to arbitrarily or completely select one of the section indicators 71 to 73 displayed around the second image selector 62, e.g., the third section indicator 73.

Specifically, the user may select one of the plurality of image selectors 61 to 63, i.e., the second image selector 62, by touching an area in which the one of the plurality of image selectors 61 to 63, e.g., the second image selector 62, is displayed, by a touching means such as a finger or a touch pen once or multiple times (tapping or double tapping).

Subsequently, the user may arbitrarily or completely select one of the plurality of section indicators 71 to 73, e.g., the third section indicator 73, by moving the touch means to one of the plurality of section indicators 71 to 73, e.g., the third section indicator 73, from the selected image selector, e.g., the second image selector 62. In this case, the user may move the touch means toward the third section indicator 73 while keeping in the touched state (dragging). In other words, the user may select one of the plurality of section indicators 71 to 73 by moving the touch means in a certain direction without separating the touch means from the touch screen 40a. In this case, the user may touch the second image selector 62 with the touch means to select the second image selector 62 and perform dragging by moving the touch means an the touch screen 40a without separating the touch means front the touch screen 40a. Furthermore, the user may additionally touch the second image selector 62 with the touch means after the second selector 62 is selected and perform dragging by moving the touch means on the touch screen 40a without separating the touch means from the touch screen 40a.

In an embodiment, the user may arbitrarily or completely select the third section indicator 73 from among the plurality of section indicators 71 to 73 by moving the touch means until the touch means reaches where the third section indicator 73 is displayed while keeping the touch means in the touched state, and in another embodiment, the user may arbitrarily or completely select the third section indicator 73 from among the plurality of section indicators 71 to 73 by moving the touch means a predefined distance or longer in the direction in which the third section indicator 73 is marked. in this case, the predefined distance may be shorter than the distance d between the section indicator 70 and the image selector.

In another embodiment, the user may touch the image selector 60 once or multiple times, and touch one of the plurality of section indicators 71 to 73, e.g., the third section indicator 73 once or multiple times after selection of the image selector 60 is completed and the plurality of section indicators 71 to 73 are displayed, thereby arbitrarily or completely selecting one of the plurality of section indicators 71 to 73.

In the same way as described above, the section indicator arbitrarily or completely selected by touching and movement, e.g., the third section indicator 73, may be displayed differently from the other section indicators, e.g., the first and second section indicators 71, 72.

Although an example of selecting the second image selector 62 and then the third section indicator 73 by inputting certain touch gestures to the touch screen 40a has been described, this may be equally or with arbitrary modification by the designer, applied to an, occasion when other image selector 61, 62 and other section indicator 71, 72 are selected.

FIG. 9B shows an example of providing information regarding a display section corresponding to a selected section indicator for the user, when one section indicator is temporarily selected.

Referring to FIG. 9B, in a case that one of the plurality of section indicators 71 to 73 is arbitrarily selected, in response to selection of the section indicator, the display 40 may provide information about a section to display an image for the user in various ways.

For example, if the third section indicator 73 is selected, the display 40 may provide information indicating that an image may be displayed in the third display section 53 corresponding to the third section indicator 73 selected from among the plurality of display sections 51 to 53. In this case, the third display section 53 corresponding to the selected third section indicator 73 may be displayed differently from the other display sections 51 and 52.

For example, a frame 53a in different color or thickness may further be displayed along the border lines of the third display section 53, or the entire or part of the third display section 53 may blink, or a certain graphic object such as a check symbol may be displayed in the entire or part of the third display section 53. Accordingly, the user may be intuitively aware of the information indicating that an image may be displayed in the third display section 53 according to the selection of the third section indicator 73. In addition, in various ways that may be considered by the designer, a display section corresponding to the selected section indicator 71 to 73 may be changed in a predetermined way or arbitrarily to provide the user with information indicating that an image may be displayed in a particular display section 51 to 53.

In this case, it may be configured such that the selected section indicator, e.g., the third section indicator 73, may be displayed differently from the other non-selected section indicators, e.g., the first and second section indicators 71 and 72. Furthermore, it may be configured such that the selected section indicator, e.g., the third section indicator 73, may be displayed identically to the other non-selected section indicators, e.g., the first and second section indicators 71 and 72 in terms of the shape or color, despite the user's selection. As described above with reference to FIGS, 6 to 9B, when one of the plurality of section indicators 71 to 73 is arbitrarily or completely selected, a predetermined image may be displayed in the display section corresponding to the selected section indicator.

Meanwhile, in an embodiment, if none of the plurality of section indicators 71 to 73 is selected by the user, the plurality of section indicators 71 to 73 may disappear after the lapse of a predetermined period of time. In other words, after the plurality of section indicators 71 to 73 are displayed, if no command to select a particular section indicator 71 to 73 is input from the user until a period of time set by the designer or the user elapses, displaying of the section indicators 71 to 73 is stopped. Even after the displaying of the section indicators 71 to 73 is stopped, if one of the image selectors 60 is selected by the user, at least one section indicator 70 may be displayed again in response to the selection of the image selector 60.

In some embodiments, the plurality of section indicators 71 to 73 may not be displayed. For example, when the user performs directional manipulation by manipulating a particular direction key, moving the cursor in a particular direction, or performing touch-and-drag operation, one of the plurality of display sections 51 to 53 corresponding to the directivity given by the user may be displayed differently from the other display sections, thereby letting the user know of which display section may display an image.

For example, if the user selects one of the image selectors 60 and enters a command to indicate a particular direction from the selected image selector 60, e.g., the upper right direction, a display section corresponding to the upper right direction, e.g., the third display section 53, may be displayed differently from the other display sections 51, 52. In this case, if the user completely selects the particular direction, e.g., the upper right direction, in a way of manipulating a mouse button or holding a touch for a certain time, a display section corresponding to the upper right direction, e.g.:, the third display section 53, displays an image.

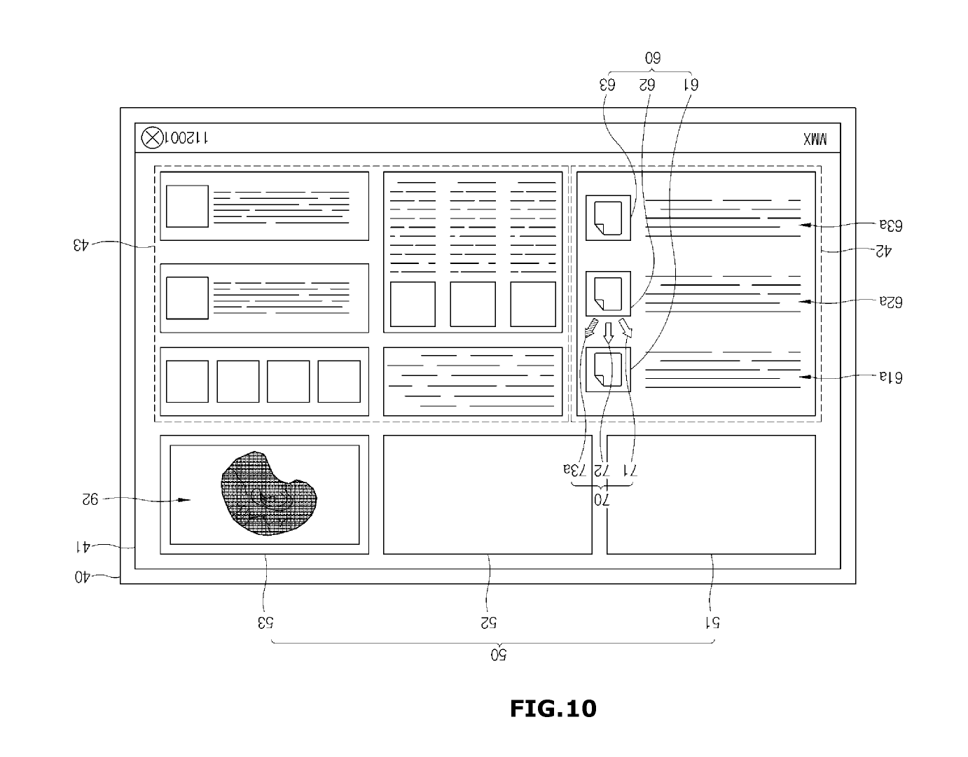

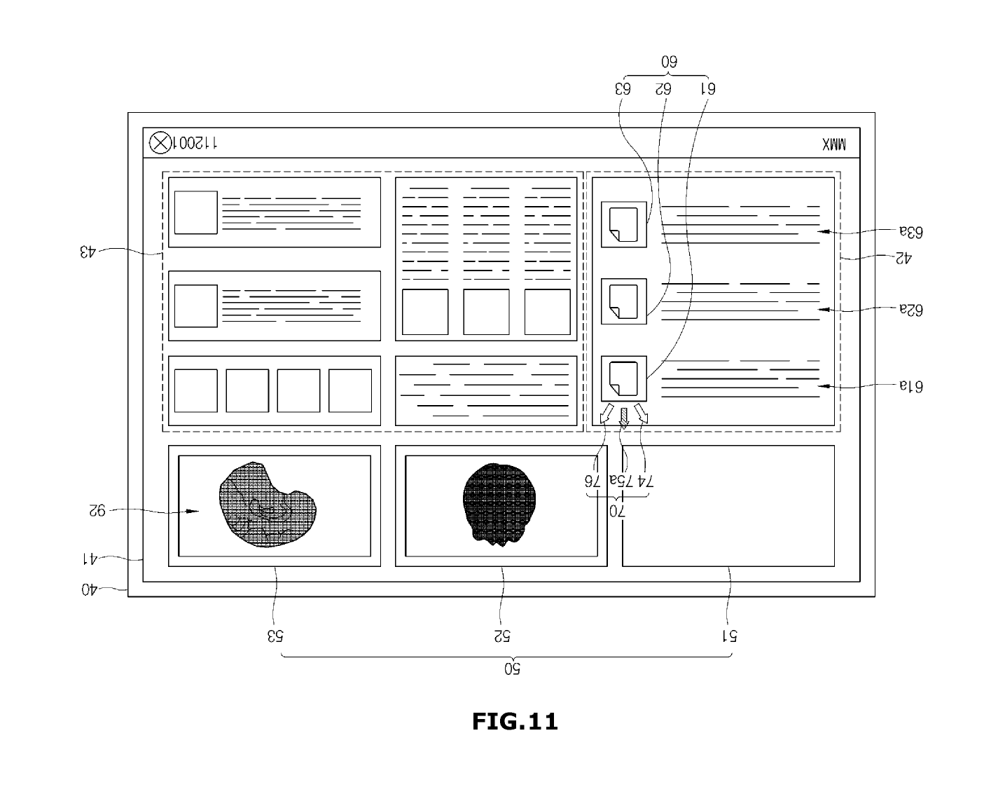

FIG. 10 shows a first example of a screen having an image displayed in a display section in response to selection of a section indicator, and FIG. 11 shows a second example of a screen having an image displayed in a display section in response to selection of a section indicator.

When one of at least one image selector 60 is selected and one of at least one section indicator displayed around the image selector is selected, an image corresponding to the selected image selector may be displayed in a display section corresponding to the selected section indicator.

For example, if the second image selector 62 is selected from among the plurality of image selectors 61 to 63 as shown in FIG. 6 and the third section indicator 73a is selected from among the plurality of section indicators 71 to 73, a display section corresponding to the third section indicator 73a, e.g., the third display section 53 may display an image called out by the second image selector 62, e.g., the second image 92. The third display section 53 may be a display section located in a direction indicated by the third section indicator 73a or arranged at a position corresponding to a position displayed around the image selector 63.

If there are a plurality of the second images 92, one of the plurality of second images 92 may be displayed according to the user's selection or predetermined settings. Furthermore, in an embodiment, a moving image may be played in the third display section 53 by sequentially displaying the plurality of second images 92 in the third display section 53.

If no image has been displayed in the third display section 53, the second image 92 may be displayed in the third display section 53, and otherwise if a particular image has been displayed in the third display section 53, the second image, may be displayed in the third display section 53 instead of the existing image. In some embodiments, the display 40 may further output a message to ask the user whether to display the second image 92 instead of the existing image, and according to the user's selection, the existing image may continue to be displayed or the second image 92 may be displayed instead of the existing image in the third display section 53.

As described above, the example of displaying an in in a display section according to selection of a section indicator may be equally or with some modification, applied to an occasion when other image selector 61, 63 is selected or other section indicator 71, 72 is selected.

When the second image 92 is displayed in the third display section 53 as shown in FIG. 11, the user may use the same method as described above to display the first image 91 in the second display section 52. Specifically, the user may have the second display section 52 display the first image 91 by selecting the first image selector 61 to call the first image 91 and selecting the second section indicator 72 corresponding to the second display section 52 from among the plurality of section indicators displayed above the first image selector 61. Accordingly, a plurality of images 92, 91 may be displayed, in the plurality of display sections 52, 53. Specifically, the first image 91 may be displayed in the second display section 52 and the second image 92 may be displayed in the third display section 53. In the same way, the user may have the first display section 51 display the third image 93.

Furthermore, in the case that the second image 92 is displayed in the third display section 53, the user may have the second display section 52 display the second image 92 by selecting the second image selector 62 again and selecting the second section indicator 72 corresponding to the second display section 52 from among the plurality of section indicators displayed above the second image selector 62. Accordingly, the second image 92 may be displayed in both the second display section 52 and the third display section 53, and in the case that the second image 92 includes a moving image, one of images forming the moving imam may be displayed in the second display section 52 and another image may be displayed in the third display section 53, allowing the user to easily compare and determine a change in the object 8 over time.

According to the method as described above, each of the plurality of display sections 51 to 53 may display a certain in age 91 to 93, and specifically, an image 91 to 93 that the riser wants to display may be easily and quickly displayed in one of the plurality of display sections 51 to 53, which is wanted by the user. Accordingly, user convenience may be improved in loading an image into a desired location.

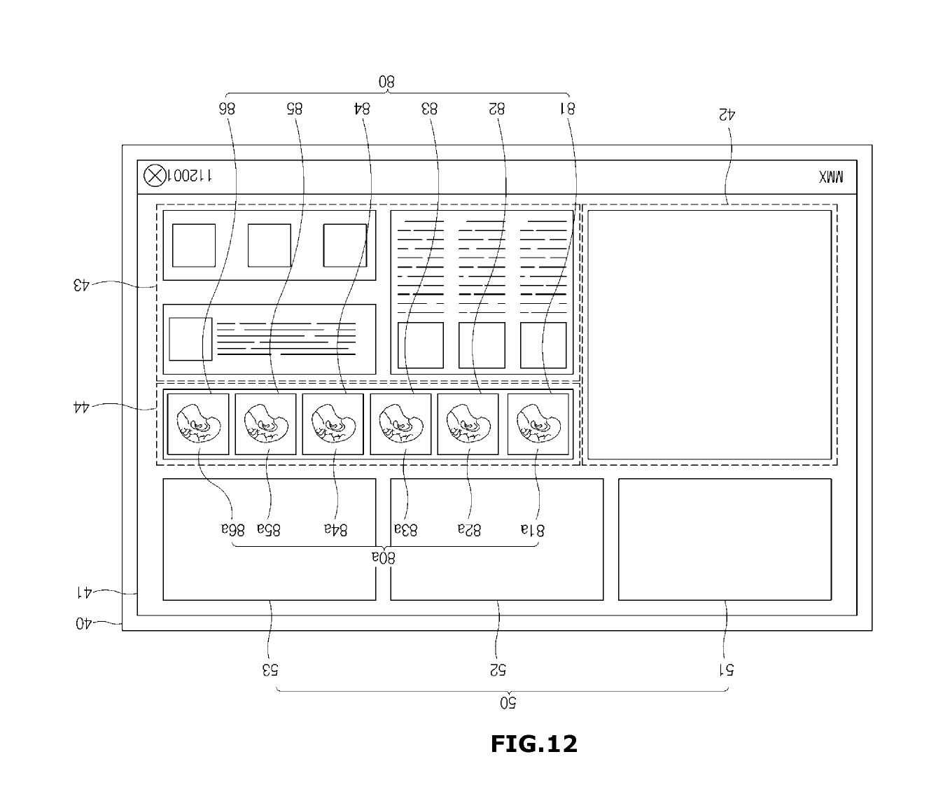

FIG. 12 shows an example of a screen with an image selector being a preview image.

In an embodiment, an image selector 80 may be implemented using at least one auxiliary image 80a corresponding to an image selected as shown in FIG. 12. The auxiliary image 80a refers to an image that may provide abstract of various information regarding the image 94. Specifically, for example, the auxiliary image 80a may include a preview image or a thumbnail image. Since the auxiliary image 80a may provide abstract of the information about the image 94 before the image 94 is completely displayed, the user may quickly obtain rough information about the image 94 to be displayed from the auxiliary image 80a.

In an embodiment, the auxiliary image 80a may be obtained by reducing the size of the original image 94 or reducing the size (volume) of a file corresponding to the original image 94. More specifically, for example, the auxiliary image 80a may be obtained by scaling dawn the width or height of the original image 94, in which case the resolution of the auxiliary image 80a may be lower than the resolution of the image 94. In some embodiments, the auxiliary image 80a may be obtained from all or part of the original image 94. In a case that the auxiliary image 80a is obtained from part of the original image 94, the auxiliary image 80a may be obtained by cutting sonic of the original image into a predetermined size. Besides, in various ways that the designer may consider, the auxiliary image 80a may be obtained from the original image 94.

An auxiliary image display section 44 may be provided on the screen 41 and an image selector 80 implemented by the auxiliary image 80a may be displayed in the auxiliary image display section 44. For example, as shown in FIG. 12, the auxiliary image display section 44 may be arranged between the display section 50 and an auxiliary display section 43. Where to arrange the auxiliary image display section 44 is not, however, limited thereto, and the auxiliary image display section 44 may be displayed anywhere on the screen 41 according to the designer's selection.

A plurality of image selectors 81 to 86 may be displayed in the auxiliary image display section 44, and the respective image selectors 81 to 86 may be implemented with auxiliary images 81a to 86a each corresponding to the image 94 to be called out according to selection of each of the image selector 81 to 86.

Each of the image selectors 81 to 86 in the auxiliary image display section 44 may receive a command to call a corresponding image 91 to 93 among the images 94 stored in the database 90 according to the user's selection. In this case, the image selectors 81 to 86 may each receive a command to call a different image 94 or receive a command to call the same image. Furthermore, some of the plurality of image selectors 81 to 86 may receive commands to call the same image and sonic other image selectors 81 to 86 may receive commands to call different images.

The respective image selectors 81 to 86 may be implemented with auxiliary images 81 a to 86a each corresponding to the image 94 called by each image selector 81 to 86. In this case, the auxiliary images 81a to 86a functioning as the respective image selectors 81 to 86 may be the same or different according to the images 94 called by the respective image selectors 81 to 86.

One of the plurality of image selectors 81 to 86, e.g., the fourth image selector 84, may be selected by the user. In this case, for example, the user may select the fourth image selector 84 in various methods, such as by touching the fourth image selector 84, manipulating the mouse device 32 to place the cursor 49 on the fourth image selector 84, or placing the cursor 49 on the fourth image selector 84 and clicking the fourth image selector 84 once or multiple tames.

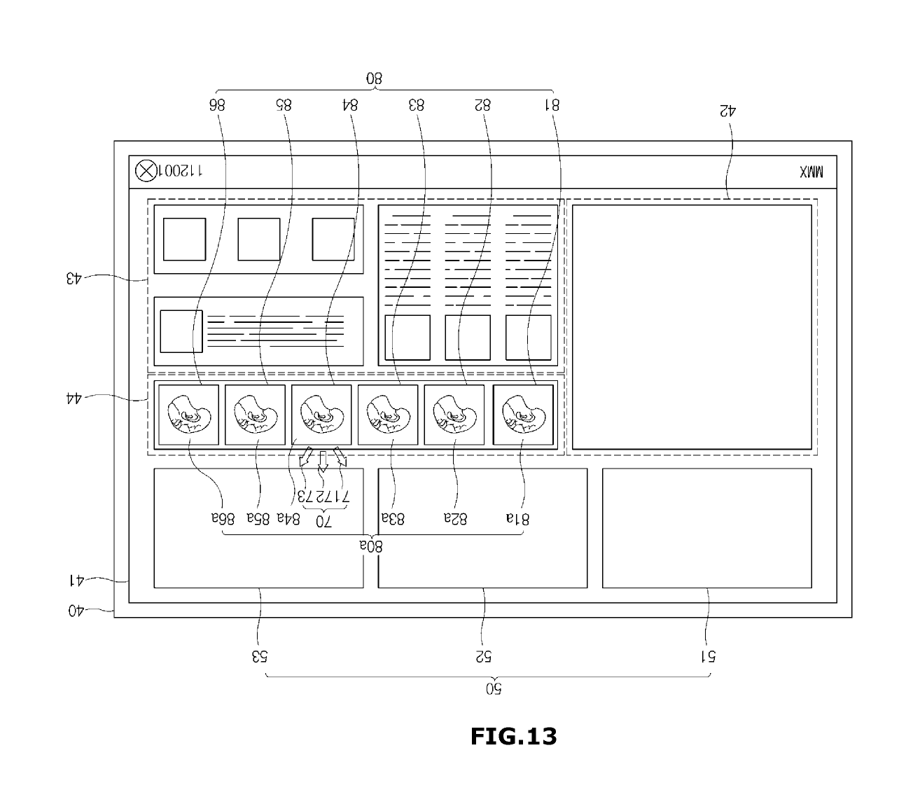

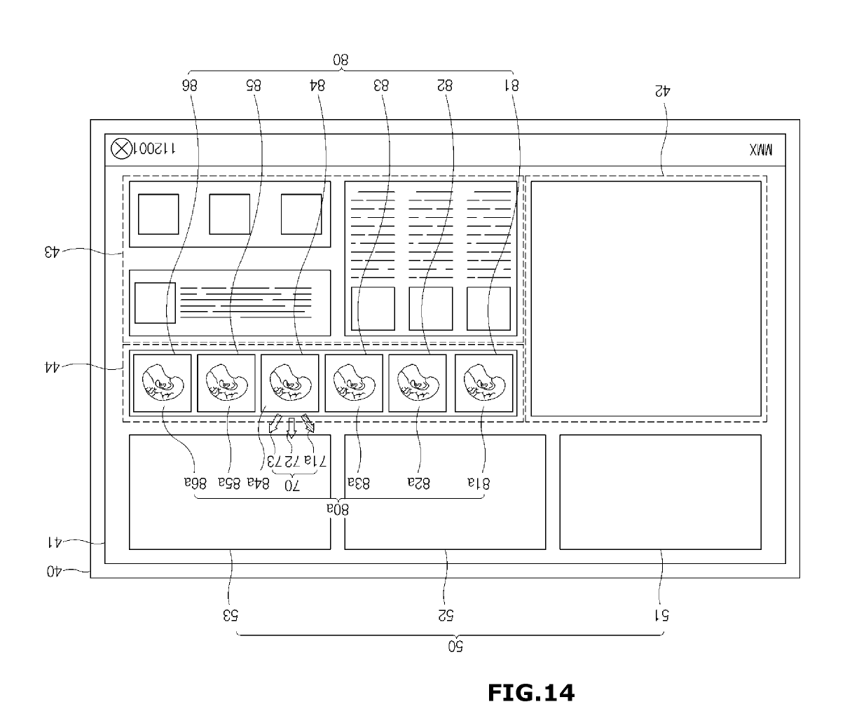

FIG. 13 shows an example of a screen with a plurality of section indicators displayed around a preview image. FIG. 14 shows an, example of a screen displayed when one section indicator is selected.

Referring to FIG. 13, in an embodiment, when one of the plurality of image selectors 81 to 86, e.g., the fourth image selector 84, is selected, at least one section indicator 70 may be displayed around the fourth image selector 84. In this case, the at least one section indicator 70 may be displayed to overlap with the fourth image selector 84 in part or as a whole, or may be displayed separately from the fourth image selector 84. In other words, the at least one section indicator 70 may be displayed to overlap with the fourth auxiliary image 84a functioning as the fourth image selector 84 in part or as a whole, or may be displayed separately from the fourth auxiliary image 84a. The at least one section indicator 70 may be provided to correspond to the display section 50 as described above. For example, if three display sections 51 to 53 are displayed on the screen 41, three or less section indicators 71 to 73 may be displayed around the fourth image selector 84. The section indicators 71 to 73 may have forms that may indicate corresponding display sections 51 to 53, such as arrow forms. Since the section indicators 71 to 73 are described above, detailed description thereof will be omitted below.

One of the displayed plurality of section indicators 71 to 73, e.g., the first section indicator 71, may be arbitrarily or completely selected according to the user's manipulation. In this case, the user may arbitrarily or completely select one of the displayed plurality of section indicators 71 to 73, e.g.,, the first section indicator 71, by making a certain touch gesture on the touch screen 40a, such as touching the first section indicator 71 once or multiple times, moving the cursor 49 onto the first section indicator 71, moving the cursor 49 onto the first section indicator 71 and clicking a button of the mouse device 32 once or multiple times, using the mouse device 32 to drag from the fourth auxiliary image 84a to the first section indicator 71, or holding touch and dragging from the fourth auxiliary image 14a to the first section indicator 71.

Once one of the displayed plurality of section indicators 71 to 73, e.g., the first section indicator 71, is arbitrarily or completely selected according to the user's manipulation, as shown in FIG. 14, the selected first section indicator 71a may be displayed differently from the other section indicators 72, 73. For example, the selected first section indicator 71a may be displayed in a different shape, size, or color from the other section indicators 72, 73. The example of selecting one of the displayed plurality of section indicators 71 to 73 or displaying the selected section indicator differently was described above, so the detailed description thereof will be omitted below.

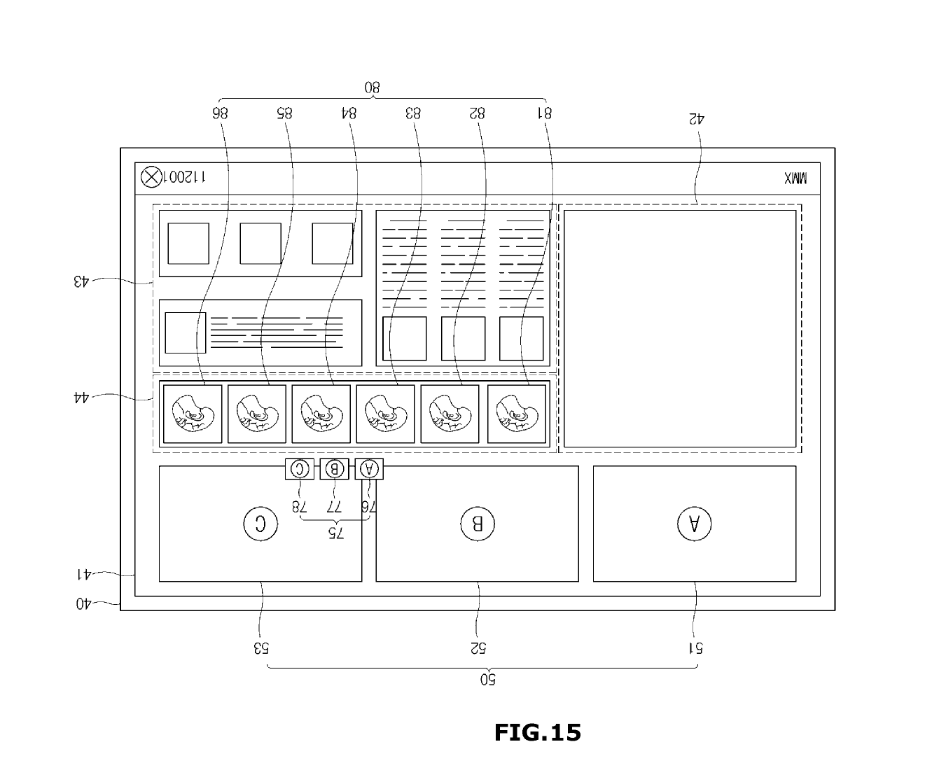

FIG. 15 shows another example of a screen displayed when one section indicator is arbitrarily or completely selected.

Referring to FIG. 15, in an embodiment, when one of the plurality of image selectors 81 to 86, e.g., the fourth image selector 84, is selected, at least one section indicator 75 implemented with an auxiliary image may be displayed around the selected image selector, i.e., the fourth image selector 84. The auxiliary image may include a thumbnail image. In the same way as described above, the at least one section indicator 75 in the form of an auxiliary image may be displayed to overlap with the fourth image selector 84 in part or as a whole or to be separated from the fourth image selector 84, according to the design of the designer.

The display 40 may display a plurality of section indicators 76 to 78 in the form of a plurality of auxiliary images, and in same embodiments, display at least one of the section indicators 76 to 78 in the form of the plurality of auxiliary images. In this case, the section indicators 76 to 78 having the form of the auxiliary images are provided to correspond to the respective display sections 51 to 53. In other words, the section indicator 76 having the form of the first auxiliary image corresponds to the first display section 51, the section indicator 77 baying the form of the second auxiliary image corresponds to the second display section 52, and the section indicator 78 having the form of the third auxiliary image corresponds to the third display section 53.

In an embodiment, at least one of the section indicators 76 to 78 each having the form of an auxiliary image displayed on the display 40 may display an image being currently displayed in the display section 51 to 53. Specifically, the section indicator 81 having the form of the first auxiliary image may transform an image being currently displayed in the first display section 51 into a smaller sire and display it in the form of an auxiliary image. Accordingly, the user may become aware of which one of the plurality of display sections 51 to 53 may display the image expected to be displayed according to the selection of the image selector 80, e.g., the fourth image selector 84 in more detail. For example, according to selection of the image selector 80, the section indicators 76 to 78 in the form of a plurality of thumbnail images may be checked out and thus, the display section to display an image may be known more intuitively. Furthermore, the user may intuitively determine which one of the currently displayed images will be replaced by the image expected to be displayed according to the selection of the image selector 80.

In an embodiment, only one section indicator in the form of an auxiliary image, e.g., the first section indicator 76, corresponding to the direction of mouse manipulation of the user or moving direction of a touch gesture, may be displayed on the display 40.

In another embodiment, the plurality of section indicators 76 to 78 in the form of a plurality of auxiliary images may be displayed on the display 40, in which case, a section indicator in the form of an auxiliary image corresponding to the direction of mouse manipulation of the user or moving direction of a touch gesture, e.g., the first section indicator 76 in the form of an auxiliary image, may be displayed in a different form from the other section indicators 77, 78 in the form of other auxiliary images. For example, a section indicator in the form of a thumbnail image corresponding to the direction of mouse manipulation of the user or moving direction of a touch gesture, e.g., the first section indicator 76 in the auxiliary image form, may be displayed to be larger than the other section indicators 77, 78 having other auxiliary image forms, or displayed while blinking, or displayed with an additional frame displayed around the first section indicator 76 of the auxiliary image form. In various other methods that may be considered by the designer, the first section indicator 76 in the form of an auxiliary image may be displayed differently from the other section indicators 77, 78 in the form of other auxiliary images. The section indicator in the form of an auxiliary image corresponding to the direction of mouse manipulation of the user or moving direction of a touch gesture may be set to a section indicator in the form of an auxiliary image located in the same or similar direction to the direction of mouse manipulation or moving direction of the touch gesture.

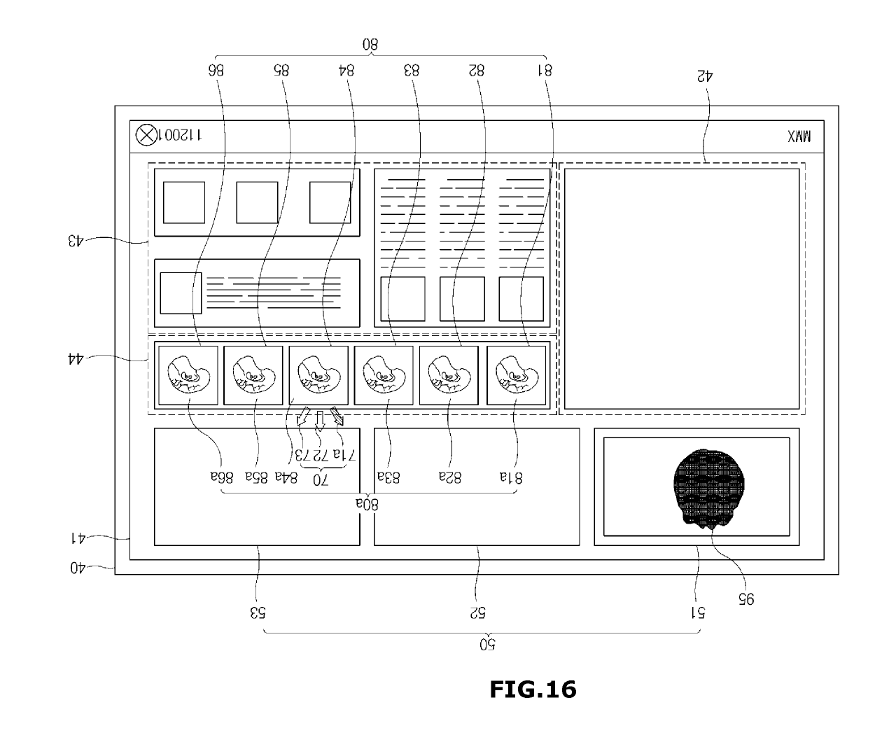

FIG. 16 shows an example of a screen having an image displayed in a display section in response to selection of a section indicator.

When one of the at least one image selector 80, e.g., the fourth image selector 84, is selected and one of the at least one section indicator 71 to 73 displayed around the selected fourth image selector 84, e.g., the first section indicator 71, is selected, as shown in FIG. 16, an image corresponding to the selected fourth image selector 84, e.g., the fourth image 95, may be displayed in a display section corresponding to the selected section indicator, e.g., the first display section 51. In the same way, the user may also have a desired image displayed in other display section 52, 53 by selecting one image selector 80 implemented with an auxiliary image 80a and one section indicator 72, 73 corresponding to the other display section 52, 53. Accordingly, the user may have a desired image easily and quickly displayed in a certain display section by using a section indicator displayed on or around an auxiliary image 80a.

Various embodiments of the medical device 1, which is an embodiment of an image displaying device, have thus far been described. The image displaying device is not, however, limited to the medical device 1. For example, the image displaying device may include a portable terminal device, such as a laptop computer, a smartphone, a tablet PC, a cellular phone, a portable game device, a personal digital assistant (PDA) device, or an electronic organizer, In this case, the respective features of the medical device 1 may be equally or with some modification, applied to the portable terminal device. Such a portable terminal device may be provided separately from the medical device 1, in which case the portable terminal device may receive various images 94 from the medical device 1 and provide the received image 94, the image selector 60, 80, and the section indicator 70 by displaying them. In the same way as in the case of the medical device 1, the user may manipulate the image selector 60, 80 and the section indicator 70 displayed in the portable terminal device to have a desired image displayed in some area on the screen of the portable terminal device. Furthermore, the image displaying device may include a general desktop computer equipped with a monitor device and an input means, e.g., a keyboard or a mouse device. The image selector 60, 80 and the section indicator 70 may be displayed on the screen of the monitor device of the desktop computer, and the user may manipulate the input means equipped in the desktop computer to select the image selector 60, 80 and the section indicator 70, thereby having various images, e.g., movies or dramas, displayed in a particular area on the screen displayed by the monitor device.

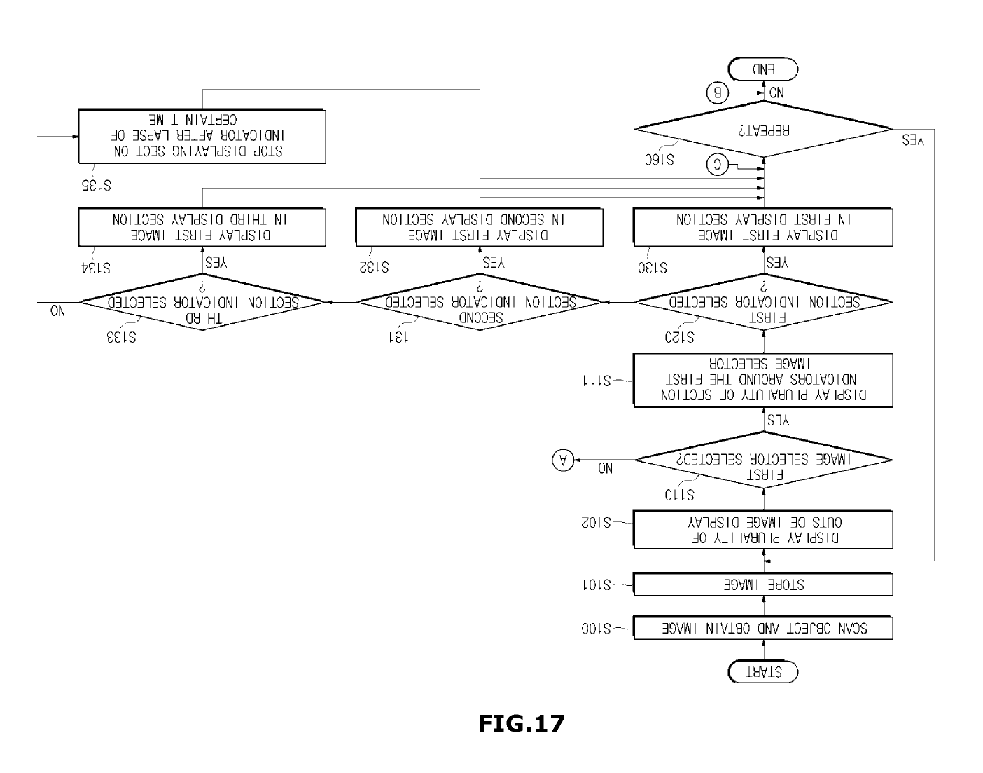

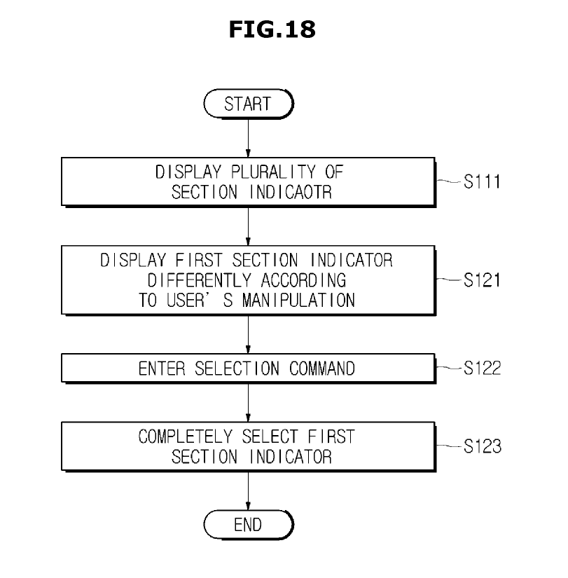

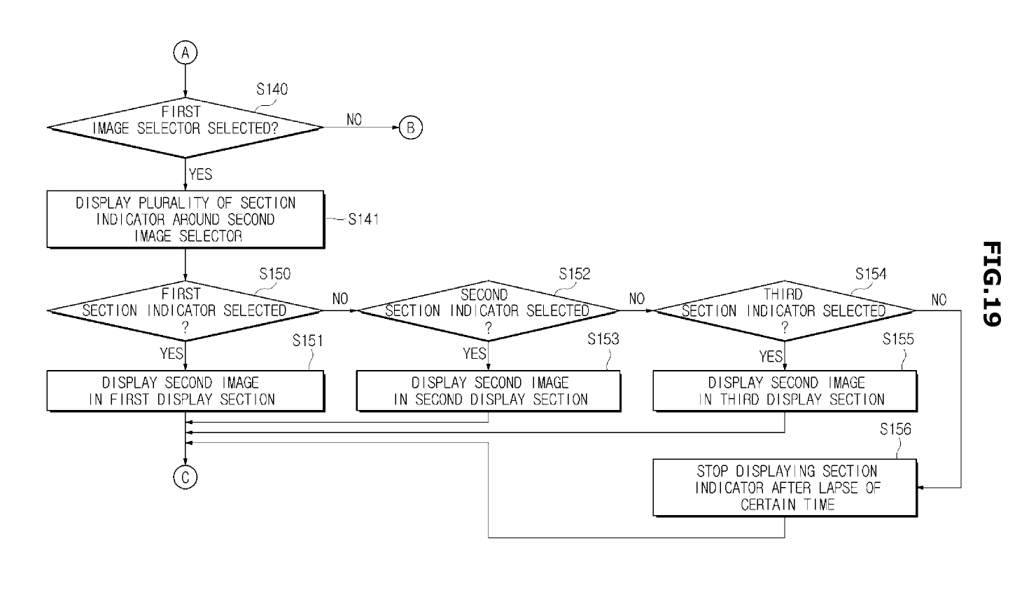

FIG. 17 is a first flowchart of a control method of an image displaying device, according to an embodiment of the present disclosure, and FIG. 18 is a second flowchart of a control method of an image displaying device, according to an embodiment of the present disclosure. FIG. 19 is a third flowchart of a control method of an image displaying device, according to an embodiment of the present disclosure.

For convenience of explanation, a control method of an image displaying device will be described based on an example in which three display sections, i.e., the first to third display sections, and three section indicators, i.e., the first to third section indicators, are provided an the screen displayed by the image displaying device.

The control method of the image displaying device is not, however, limited thereto, and may be equally or with some modification, applied to occasions when there are fewer or more display sections, when there are fewer or more image selectors, and/or when there are fewer or more section indicators.

As shown in FIG. 17, in an embodiment of a control method, of an image displaying device, an object is first scanned to obtain an image of the object, in s100. In this case, if the image displaying device is a medical device, images of an internal or external portion of the object may be obtained in various methods depending on the type of the medical device. For example, if the medical device is an ultrasonic imaging device, an ultrasonic image of the object may be obtained by an ultrasonic probe irradiating ultrasounds to the internal portion of the object and receiving ultrasounds reflecting off from the internal portion of the object, and by a main body of the ultrasonic imaging device time-delaying and concentrating the received ultrasounds.

The obtained image may be temporarily or non-temporarily stored in a storage device, in s101.

The image displaying device may display one or more image selectors on the screen, in s110. The one or more image selectors are provided to call and display the respective corresponding images on the screen, and when the user selects one of the image selectors, an image corresponding to the selected image selector is ready to be displayed on the screen. The one or more image selectors may include at least one of letters, symbols, figures to stand for the selected image, and auxiliary images corresponding to the selected image.

The user may select the first image selector from among the one or more image selectors, in s110 (yes). For example, the user may select the first image selector from among the one or more image selectors in various methods, such as moving the cursor onto the first image selector, or moving the cursor and clicking or double-clicking, or making a touch gesture.

If the user selects the first image selector from among the one or more image selectors, in s110 (yes), the image displaying device may display one or more section indicators, three section indicators, around the selected first image selector, in s111. In this case, the section indicators may be implemented using graphic objects such as arrow forms or thumbnail images. The thumbnail images may be obtained by scaling down the existing images being displayed in the respective display sections.

In some embodiments, the section indicators may not be displayed, in which case, as will be described below, it is possible to display only one section indicator at a position corresponding to a direction of the touch gesture of the user or a moving direction of the cursor according to use, of the mouse device. In this case, the single section indicator may be implemented using a graphic object such as an arrow form or an auxiliary image such as a thumbnail image.

The number of the section indicators may be the same as or be fewer than the number of the display sections. The three section indicators, in an embodiment, may each be shaped to indicate a direction in which the corresponding display section of the three display sections is located. In displaying the section indicator, the image displaying device may display the entire or part of the section indicator to overlap with the first image selector or to be a distance away from the first image selector. The three section indicators may be displayed at predetermined positions around the image selector, in which case the three section indicators may each be displayed at a position around the image selector according to a position where a corresponding display section is placed. For example, if the corresponding display section is placed in the left of the screen, the section indicator may be displayed on the left side of the image selector.

The user may select at least one section indicator, in s120, s131, s133. For example, the user may select at least one of the plurality of section indicators by placing the cursor on the at least one section indicator, select at least one of the plurality of section indicators by touching or clicking the at least one section indicator, select at least one of the plurality of section indicators by manipulating an input means, e.g., a direction key, corresponding to a section indicator, or select at least one of the plurality of section indicators by making a gesture in the direction from the image selector to the at least one section indicator. The user may also select a section indicator in a combination thereof.

If the at least one section indicator is completely or arbitrarily selected, the selected section indicator may be displayed differently from other section indicators.

Specifically, referring to FIG. 18, once a plurality of section indicators are displayed s111, one of the plurality of section indicators, e.g., the first section indicator, may be arbitrarily or completely selected by the user's selection. The first section indicator arbitrarily or completely selected may be displayed differently from the other section indicators.

For example, the user may move the cursor or focus onto a section indicator by manipulating an input means such as a mouse device or a keyboard, and when the cursor or focus is placed to overlap with the section indicator, the section indicator may be arbitrarily selected. In this case, the section indicator arbitrarily selected may be displayed differently from the other section indicators, in s121.

The user may enter a selection command by manipulating the input means, such as a button of the mouse device or keyboard in s122, and accordingly, the first section indicator may be completely selected in s123. In this case, the first section indicator may be displayed differently from the other section indicator, and if necessary, may be displayed differently from an occasion when it is arbitrarily selected.

If the user selects the first section indicator in the aforementioned method, in s120 (yes), the image displaying device may display a first image that may be selected by the first image selector in the first display section corresponding to the first section indicator among the at least one display sections, in s130. in this case, the first display section corresponding to the first section indicator may be displayed differently from the other display sections. For example, a frame in different color or thickness may further be displayed along the border lines of the first display section, or the entire, or part of the first display section may blink, or a certain graphic object may be displayed in the entire or part of the first display section.

If the user selects not the first section indicator in s120 (no) but the second section indicator in s131 (yes), the image displaying device may display the first image corresponding to the first image selector in the second display section corresponding to the second section indicator in s132. In the same way as described above, the second display section corresponding to the second section indicator may be displayed differently from the other display sections, e.g., the first and third display sections, in various ways.

If the user selects not the first and second section indicators in s131 (no) but the third section indicator in s133 (yes), the image displaying device may display the first image selected by the first image selector in the third display section corresponding to the third section indicator in s134. Even in this case, in the same way as described above, the fourth display section corresponding to the fourth section indicator may be displayed differently from the other display sections, e.g., the first and third display sections, in various ways.

If the user does not select any section indicator until a certain period of time elapses in s133 (no), the displaying of the section indicator may be stopped after the lapse of the certain period of time. In other words, the section indicator may disappear from the screen, in s135.

Meanwhile, as shown in FIG. 19, if the first image selector is not selected in s110 (no) but the second image selector is selected in s140 (yes), one or more section indicators, e.g., three section indicators, may be displayed around the second image selector in s141.

Once the three section indicators are displayed, the user may select the first section indicator in s150, or select the second section indicator in s152, or select the third section indicator in 154.

If the first section indicator is selected in s150 (yes), the second image called out using the second image selector is displayed in the first display section corresponding to the first section indicator in s151, if the second section indicator is selected in s152 (yes), the second image corresponding to the second image selector is displayed in the second display section corresponding to the second section indicator in s153, and if the third section indicator is selected in s154 (yes), the second image corresponding to the second image selector is displayed in the third display section corresponding to the third section indicator in s155. If no section indicator is selected until a certain period of time elapses in s154 (no), the displaying of the section indicator may be stopped in s156.

If the user wants to further display a desired image in other display section than the selected display section, the steps 102 to 156 may be repeated in s160.

Furthermore, if the user wants to replace an image displayed in the selected display section, e.g., the first image called out using the first image selector with another image, e.g., the second image called out using the second image selector, the steps s102 to s156 may be repeatedly performed in s160.

Accordingly, the user may have a plurality of images displayed a plurality of display sections as intended.

The control method of an image displaying device in accordance with the aforementioned embodiments may be implemented in the form of a program that may be carried out by various computer devices. The program herein may include program instructions, data files, data structures, etc., alone or in combination. The program may be designed and produced using not only machine language codes which may be made by a compiler but also high-level language codes which are executable by a computer using an interpreter. The program may be specially designed to implement the control method of an image displaying device, and may be implemented using various usable functions or definitions known to ordinary skilled people in computer software applications.

The program to implement the control method of an image displaying device may be recorded on a computer-readable recording medium. The computer-readable recording medium may include various types of hardware devices that may store particular programs that are executed by calls from computers, magnetic disk storage media like hard disks or floppy disks, magnetic tapes, optical media like compact discs (CDs) or digital versatile disks (DVDs), magneto-optical media like floptical disks, or semiconductor storage devices like read only memories (ROMs), random access memories (RAMs), or flash memories.

Although various embodiments of an image displaying device, control method thereof, and medical device are described above, the image displaying device, control method thereof, and medical device is not limited thereto, Various other embodiments that may be implemented by ordinary skilled people in the art modifying and changing the aforementioned embodiments may also fall within the scope of the present disclosure. For example, the aforementioned method may be performed in different order, and/or the aforementioned systems, structures, devices, circuits, etc., may be combined in different combinations from what is described above, and/or replaced or substituted by other components or equivalents thereof, to obtain appropriate results.

1. A control method of an image displaying device comprising:
displaying an image selector to select an image;
displaying a plurality of section indicators around the image selector;
selecting at least one section indicator from among the plurality of section indicators;
displaying an image corresponding to the image selector in a display section corresponding to the at least one section indicator.

2. The control method of claim 1, wherein the displaying of the plurality of section indicators around the image selector comprises displaying at least one of the plurality of section indicators around the image selector according to a position at which at least one display section corresponding to the at least one section indicator is placed.

3. The control method of claim 1, wherein the displaying of the plurality of section indicators around the image selector comprises displaying the plurality of section indicators around the image selector when the image selector is selected.

4. The control method of claim 1, wherein the selecting of the at least one section indicator from among the plurality of section indicators comprises displaying the at least one section indicator selected from among the plurality of section indicators differently from at least one other section indicator.

5. The control method of claim 1, wherein the displaying of the image selector to select an image comprises displaying a plurality of image selectors to select different images.

6. The control method of claim 1, wherein the image selector comprises at least one of letters, symbols, figures to stand for the selected image, and auxiliary images corresponding to the selected image.

7. The control method of claim 1, wherein the section indicator is shaped to indicate a direction in which a corresponding one of the plurality of display sections is located.

8. The control method of claim 1, wherein the selecting of the at least one section indicator from among the plurality of section indicators comprises at least one of selecting at least one of the plurality of section indicators by placing a cursor on the at least one section indicator, selecting at least one of the plurality of section indicators by touching or clicking the at least one section indicator, selecting at least one of the plurality of section indicators by manipulating an input means corresponding to each section indicator, and selecting at least one of the plurality of section indicators by making a gesture in a direction from the image selector to the at least one section indicator.

9. The control method of claim 1, wherein the selecting of the at least one section indicator from among the plurality of section indicators comprises arbitrarily selecting at least one of the plurality of section indicators and entering a command to select the at least one section indicator arbitrarily selected.

10. The control method of claim 1, wherein the plurality of display sections comprises sections capable of displaying at least one scout view.

11. An image displaying device comprising:
a display configured to display an image selector to select an image and a plurality of section indicators around the image selector; and
controller configured to control the display to display an image selected by the image selector in a display section corresponding to at least one of the plurality of section indicators when the at least one section indicator is selected.

12. The image displaying device of claim 11, wherein the display is configured to display at least one of the plurality of section indicators around the image selector according to positions at which at least one display section corresponding to the at least one section indicator is placed.

13. The image displaying device of claim 11, wherein the display is configured to display the plurality of section indicators around an image selector when the image selector is selected.

14. The image displaying device of claim 11, wherein the display is configured to display at least one section indicator selected from among the plurality of section indicators differently from at least one of non-selected section indicator.

15. The image displaying device of claim 11, wherein the display is configured to display a plurality of image selectors to select different images.
